(19) United States
(12) Patent Application Publication
(10) Pub. No.: US 2010/0324295 A1
(43) Pub. Date: Dec. 23, 2010

(54) PROCESS FOR THE PRODUCTION OF SUBSTITUTED 5-QUINOLYL-OXAZOLES AND PHARMACEUTICALLY ACCEPTABLE SALTS THEREOF

(75) Inventors: Timothy D. Cutarelli, Clinton, NJ (US); Dimitar L. Filipov, San Francisco, CA (US); Christopher Stanley Pridgen, Union City, NJ (US); Michael R. Reeder, Skillman, NJ (US); Kelvin H. Yong, Lyndhurst, NJ (US); Dimitrios N. Zarkadas, Fanwood, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/668,192

(22) PCT Filed: Jul. 3, 2008
(86) PCT No.: PCT/US08/08261

§ 371 Date: Jan. 7, 2010

Related U.S. Application Data
(60) Provisional application No. 60/959,252, filed on Jul. 10, 2007.

(51) Int. Cl. C07D 413/14; C07D 413/02

(52) U.S. Cl. 546/176

(57) ABSTRACT

The present invention relates a process for the preparation of a compound of the formula:

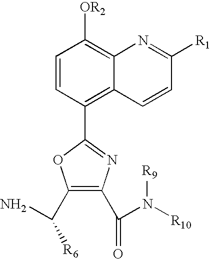

wherein R₁, R₂, R₆, R₉ and R₁₀ are as described herein. The compounds are inhibitors of phosphodiesterase 4 (PDE4).

### CROSS-REFERENCE TO RELATED APPLICATIONS

This application is commencing national stage examination pursuant to 35 U.S.C. §371 from International patent application No. PCT/US2008/008261 filed in the U.S. PCT receiving office on Jul. 3, 2008, which international application is based on and claims the priority of U.S. provisional patent application Ser. No. 60/959,252 filed Jul. 10, 2007. Each of the aforementioned PCT and Provisional applications is incorporated in its entirety by reference as if fully set forth herein.

### FIELD OF THE INVENTION

The present invention relates to processes for the preparation of intermediates useful in the preparation of substituted 5-quinolyl-oxazole and pharmaceutically acceptable salts thereof, which have utility as phosphodiesterase inhibitors.

### BACKGROUND OF THE INVENTION

Identification of any publication, patent, or patent application in this section or any section of this application is not an admission that such publication is prior art to the present invention.

Phosphodiesterases are known to regulate cyclic AMP, and phosphodiesterase 4 (PDE4) has been shown to be the predominant regulator of cyclic AMP in respiratory smooth muscle and inflammatory cells. Inhibitors of PDE4 are useful in treating a variety of diseases, including allergic and inflammatory diseases, diabetes, central nervous system diseases, pain, and viruses that produce TNF.

Amino-substituted quinolyl PDE4 inhibitors are disclosed in U.S. Pat. No. 5,804,588; sulfonamide-substituted quinolyl PDE4 inhibitors are disclosed in U.S. Pat. No. 5,834,485; and (benzo-fused)heteroaryl-substituted PDE4 inhibitors are disclosed in U.S. Pat. No. 6,069,151. Oxazolyl-substituted quinolyl PDE4 inhibitors are disclosed in PCT/US2005/017134.

A process for the production of substituted 5-quinoly-oxazoles, for example, the compounds of Formulae I and II,

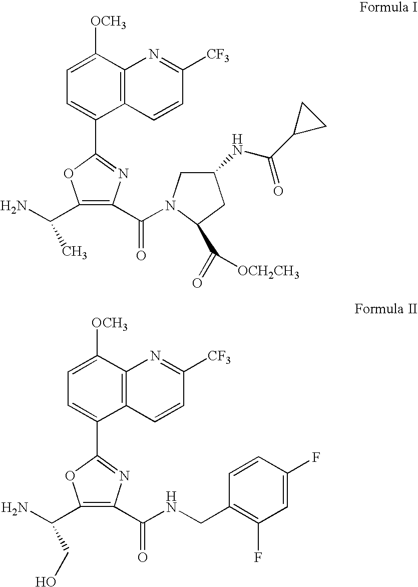

is described in US 2006/0106062 A1, published May 18, 2006 (the '062 publication), which application is incorporated herein by reference in its entirety. In particular, the '062 publication describes a batch process for the preparation of the compound of Formula I on pages 83 to 86 (preparative Examples 5 to 7, in preparation of the example compound 26-381 (which is illustrated on page 204 of the '062 publication), which is illustrated on page 193) and a process for the preparation of the compound of Formula II on pages 380 to 382 (preparative Examples 8 to 11 in preparation of the example compound 38-8, which is illustrated on page 383), each of which process is incorporated by reference herein in its entirety.

In particular, the compound of Formula I has been prepared in accordance with Scheme Ia.

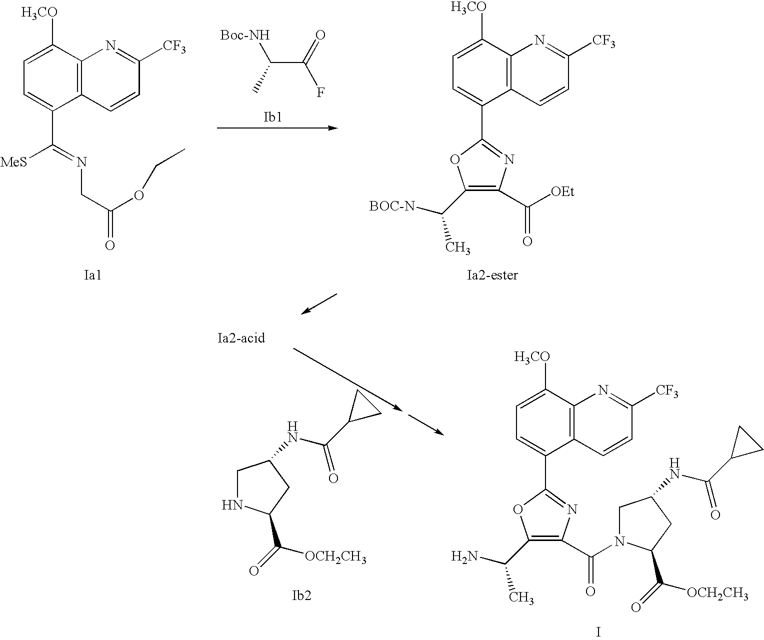

With reference to Scheme Ia, preparation of the trisubstituted oxazole of Formula Ia2-ester, by treatment of the compound of Formula Ia1 with the acid halide of Formula Ib1, and of the compound of Formula Ia2-acid prepared by hydrolyzing the ester functional group of the oxazole of Formula Ia2-ester, provides a product which contains high levels of unwanted side reaction products and shows poor utilization of the starting materials. Moreover, it is desirable to provide a salt form of the compound of Formula I in the preparation of an inhaled medicament, particularly when the medicament comprises a combination with additional pharmaceutically active compounds. Heretofore, no reliable means of providing a salt of the compound of Formula I having an acceptable particle size range mean particle size and low amorphous material content suitable for inclusion in a medicament for inhalation administration has been available.

### OBJECTIVES AND SUMMARY OF THE INVENTION

What is needed is a process for the provision of trisubstituted oxazole compounds, particularly the compound of Formula I, which is amenable to preparation of commercial scale quantities of the compounds, has acceptable utilization of the starting materials, and reliably provides a salt of the compound having acceptable particle size distribution and acceptably low amorphous material content. These and other objectives are met by the present inventive process which in one aspect is a process for preparing a compound of Formula I in accordance with Scheme Ia2

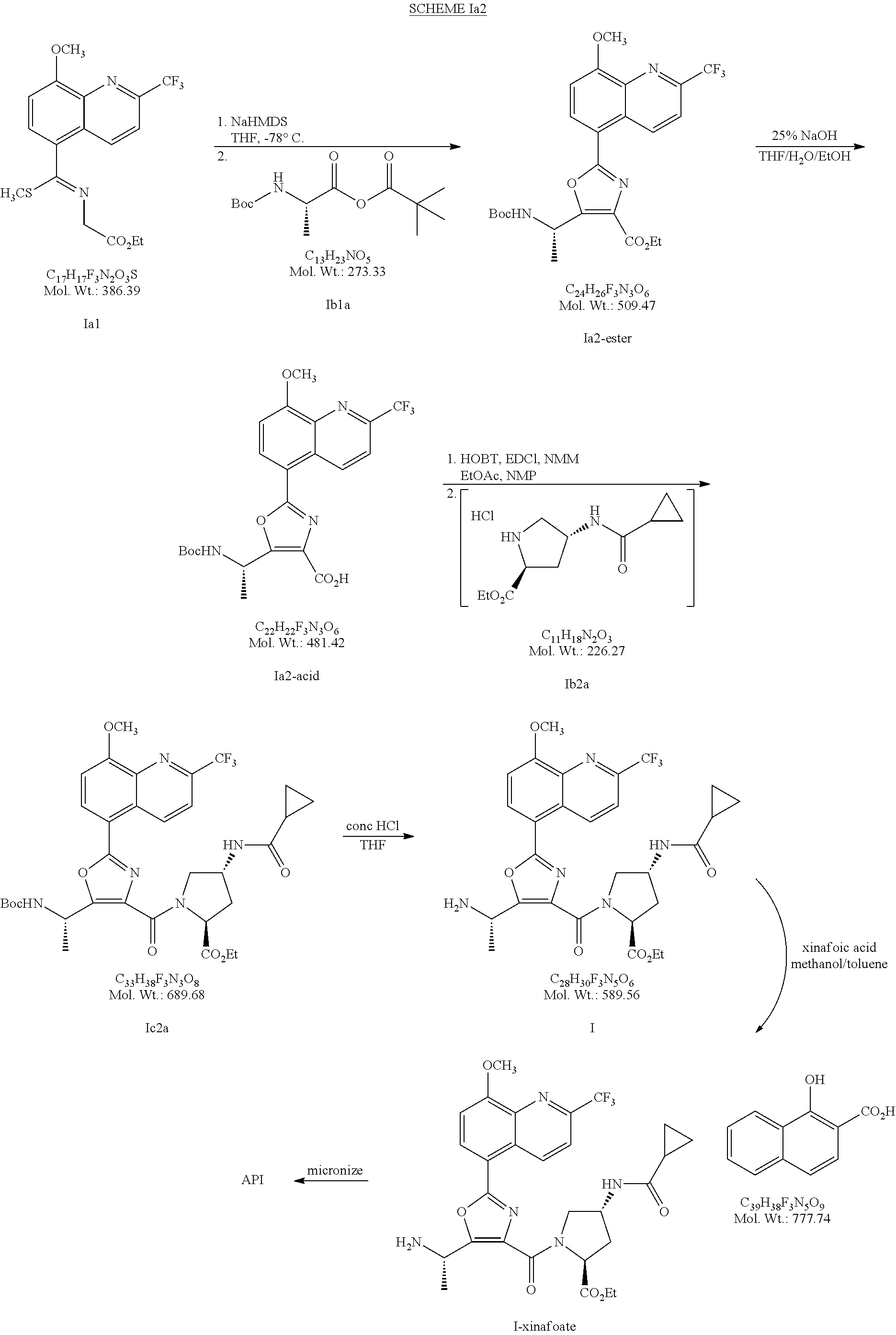

the process comprising:
(a) providing the anhydride of Formula Ib1a;
(b) reacting the compound of the Formula Ia1 with an alkali metal amide base, preferably sodium bis(trimethylsilyl)amide (NaHMDS), and reacting that product with the anhydride provided in Step “a” to provide the compound of Formula Ia2-ester;
(c) converting the compound of Formula Ia2-ester to the acid of Formula Ia2-acid by treatment with aqueous base;
(d) reacting the compound of Formula Ia2-acid with the amino salt compound of the Formula Ib2a to form the compound of Formula Ic2a;
(e) deprotecting the compound of Formula Ic2a to form the compound of Formula I;
(f) optionally precipitating the xinafoate salt of the compound of Formula I by treatment with xinafoic acid to provide a compound of the Formula I-xinafoate;
(g) optionally, when optional step “f” has been carried out, drying the precipitated salt in an agitated dryer under the following regime: (a) Tⱼ=50° C., no agitation for 3 hours at 0.1 bar pressure; (b) Tⱼ=80° C., agitation set at 20 rpm for 12 hours at 0.1 bar pressure; and (c) Tⱼ=80° C., agitation set at 60 rpm for 12 hours at 0.1 bar pressure, wherein Tⱼ is the jacket temperature of the rotary dryer used to dry the compound; and
(h) optionally micronizing the compound of Formula I-xinafoate to provide an active pharmaceutical ingredient comprising the compound of Formula I.

In some embodiments it is preferred to carry out the provision of the anhydride of Formula Ib1a in step “a” by a process comprising reacting the compound of Formula Ib1b (N-Boc-L-alanine),

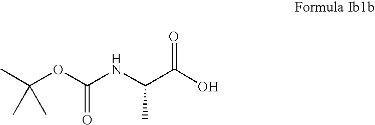

with trimethylacetylchloride in the presence of dicyclohexylamine.

In some embodiments of the inventive process, step “b” is carried out using incremental addition of alkali metal amide base and anhydride, thus, step “b” is carried out by placing the compound of Formula Ia1 in a reaction mixture, adding an aliquot of an alkali metal amide base in an amount which is less than required to react with all of the compound of Formula Ia1 present, then add an equivalent amount of the anhydride compound of Formula Ib1a, and repeat the addition of amide followed by anhydride until substantially all of the compound of Formula Ia1 has been reacted. In some embodiments using incremental addition in Step “b” of the inventive process, it is preferred to select an aliquot size of the alkali metal amide base such that the amount of alkali metal amide base needed to complete the reaction is added in 10 separate aliquots, each of which is followed by the addition of an appropriate amount of anhydride.

In some embodiments of the inventive process, step “b” is carried out using continuous streams of the compound of Formula Ia1 and alkali metal amide base (amide base), which are mixed in a static mixer and quenched in a quenching vessel containing the anhydride compound of Formula Ib1a. In some embodiments of the inventive process utilizing continuous streams of the compound of Formula Ia1 and amide to carry out step “b” of the process it is preferred to carry out the reaction at a temperature of less than about 0° C., preferably less than about [−25° C.], more preferably less than about [−50° C.], and more preferably less than about [−75° C.].

In some embodiments of the inventive process it is preferred to provide the compound of Formula Ib2a by deprotecting the t-BOC-N protected precursor of Formula Ib2a1 provided by reacting the compound of Formula Ib2a1 in ethyl-acetate with gaseous HCl.

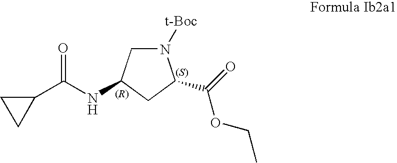

In another aspect the present invention is a process for preparing an oxazole compound of Formula ID:

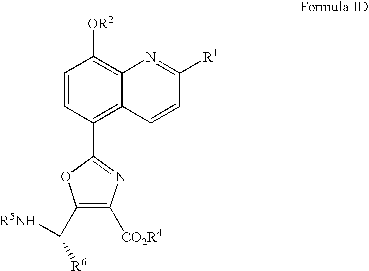

wherein
   R¹ is a haloalkyl;
   R², R⁴ are selected independently and are alkyl;
   R⁵ is an acid labile amino protecting group; and
   R⁶ is hydrogen, methyl, alkyl of 2 carbons or more, hydroxyalkyl, alkoxyalkyl, mercaptoalkyl, —CH₂F, —CHF₂, —CF₃, —C(O)OH, —C(O)Oalkyl or —C(O)NR⁴³R⁴⁴, wherein R⁴³ and R⁴⁴ are independently H or alkyl, preferably R⁶ is H or methyl;

the process comprising:
(a) providing a stream comprising the compound of Formula IDa,

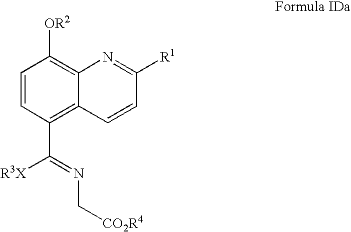

wherein
   R¹, R² and R⁴ are defined above;
   R³ is selected from hydrogen and alkyl; and
   X is selected from oxygen and sulfur;
(b) reacting the stream provided in Step “a” with an alkali metal amide base; and
(c) quenching the reaction mixture from step “b” in a quenching medium comprising a compound of Formula IDb,

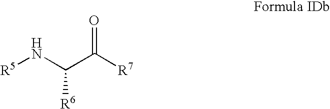

wherein
   R⁵ and R⁶ are as defined above; and
   R⁷ represents an acid activating moiety.

In some embodiments it is preferred to carry out the oxazole-producing process using a compound of the Formula IDb wherein R⁷ is a halogen, an alkylcarbonyloxy moiety, a morpholino moiety of the formula:

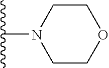

or a derivative thereof, or an imidazole moiety of the formula:

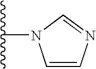

or a derivative thereof, preferably R⁷ is a halogen or an alkylcarbonyloxy moiety, more preferably R⁷ is an alkylcarbonyloxy moiety. In some embodiments it is preferred to carry out the oxazole-producing process of less than about 0° C., preferably at a temperature of less than about [−25° C.], more preferably at a temperature of less than about [−50° C.]. and more preferably at a temperature of less than about [−75° C.].

Other aspects and advantages of the present invention will become apparent from following Detailed Description.

### BRIEF DESCRIPTION OF THE DRAWINGS

FIG. 1 is a schematic flow diagram of an apparatus for conducting a continuous reaction process according to an embodiment of the present invention.

### DETAILED DESCRIPTION

As mentioned above, processes for the preparation of various oxazoles having PDE 4-inhibiting properties have been described, including those described in U.S. patent application Ser. No. 11/775,383 filed on Jul. 10, 2007, which application is incorporated herein in its entirety by reference.

In some aspects, substituents referenced herein have been defined in terms of the definitions for certain identified substituents presented in published U.S. Patent Publication No. 2006/0106062 (the '062 publication), for example, the substituents identified as R⁷ and R⁸: in the '062 publication, which are referenced in the definition of certain substituents defined herein, have the following definitions in the '062 publication:
R⁷ is H, alkyl, alkenyl, hydroxyalkyl, cycloalkyl, alkoxyalkyl, aminoalkyl, (R¹⁷-phenyl)alkyl or —CH₂—C(O)—O-alkyl;
R⁸ is H, alkyl, alkenyl, alkoxy, alkoxyalkyl, hydroxyalkyl, dihydroxyalkyl, alkyl-NR¹⁸R¹⁹, cyanoalkyl, haloalkyl, R²³-heteroaryl, R²³-heteroarylalkyl, R³⁶-heterocycloalkyl, (R³⁶-heterocycloalkyl)alkyl, R¹⁷-phenyl, (R¹⁷-phenyl)alkyl, R¹⁷-naphthyl, (R¹⁷-naphthyl)alkyl, R¹⁷-benzyloxy, -alkyl-C(O)—NR¹⁸R¹⁹, -alkyl-C(O)—N(R³⁰)—(R²³-heteroaryl), -alkyl-C(O)—(R¹⁷-phenyl), -alkyl-C(O)—(R³⁶⁻heterocycloalkyl);)-alkyl-N(R³⁰)—C(O)Oalkyl, -alkyl-N(R³⁰)—C(O)—NR¹⁸R¹⁹, -alkyl-N(R³⁰)—C(O)alkyl,)-alkyl-N(R³⁰)—C(O)-(fluoroalkyl), -alkyl-N(R³⁰)—C(O)—(R³⁹-cycloalkyl), -alkyl-N(R³⁰)—C(O)—(R¹⁷-phenyl), -alkyl-N(R³⁰)—C(O)—(R²³-heteroaryl), -alkyl-N(R³⁰)—C(O)-alkylene-(R²³-heteroaryl), -alkyl-NH—SO₂—NR¹⁸R¹⁹, -alkyl-N(R³⁰)—(R¹⁷-phenyl),)-alkyl-N(R³⁰)—(R²³-heteroaryl), -alkyl-O—(R¹⁷-phenyl), -alkyl-O—(R²³-heteroaryl), -alkyl-N(R³⁰)—SO₂-alkyl, alkylthioalkyl-, alkyl-SO₂-alkyl-, (R³⁵-phenylalkyl)-S-alkyl-, (hydroxyalkyl)-S-alkyl-, (alkoxyalkyl)-S-alkyl-, -alkyl-CO₂-alkyl, R⁴⁶-hydroxyalkyl, dihydroxyalkyl substituted by R¹⁷-benzyloxy, dihydroxyalkyl substituted by R¹⁷-phenyl, alkoxyalkyl substituted by R¹⁷-phenyl, (R¹⁷-phenyl)alkyl substituted by —CO₂alkyl, (R¹⁷-phenyl)alkyl substituted by —C(O)N(R³⁰)₂, alkyl substituted by (R²³-heteroaryl) and —C(O)NR³⁷R³⁸, haloalkyl substituted by CO₂alkyl, R¹²-cycloalkyl, (R¹²-cycloalkyl)alkyl,

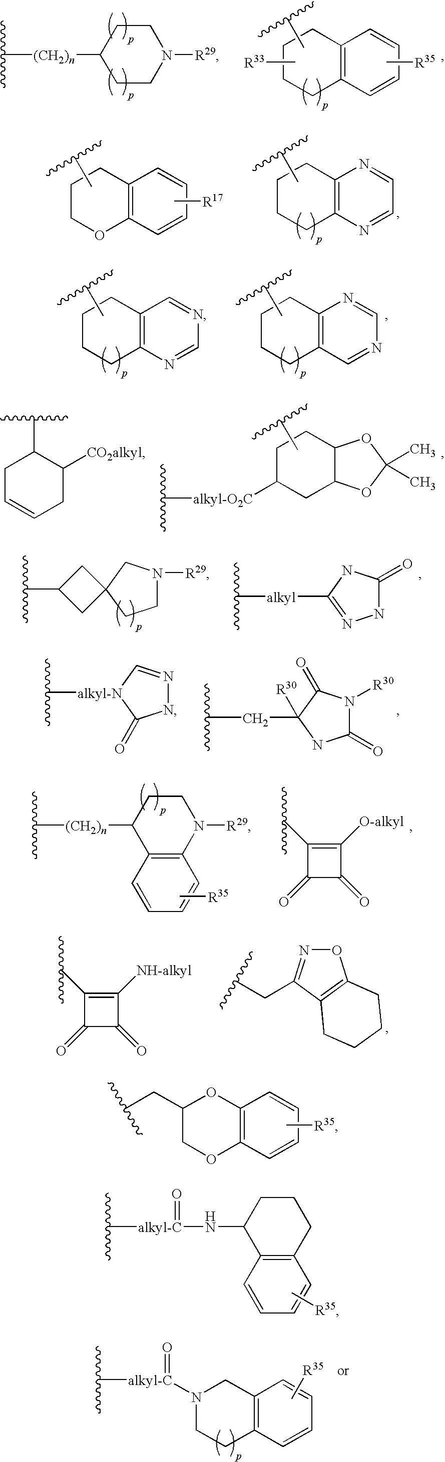

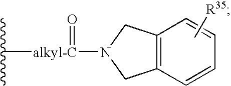

or R⁷ and R⁸ and the nitrogen to which they are attached together form a ring system selected from the group consisting of

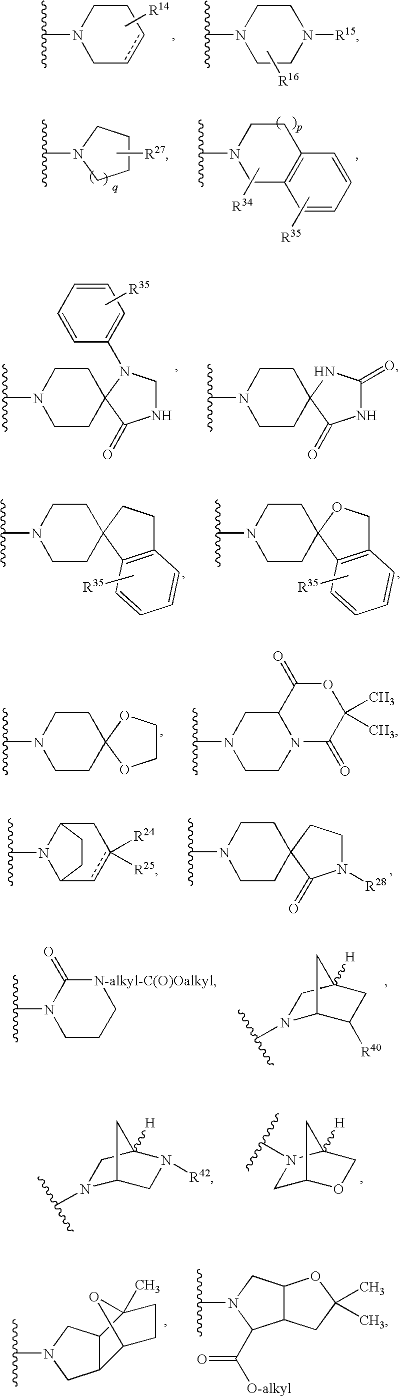

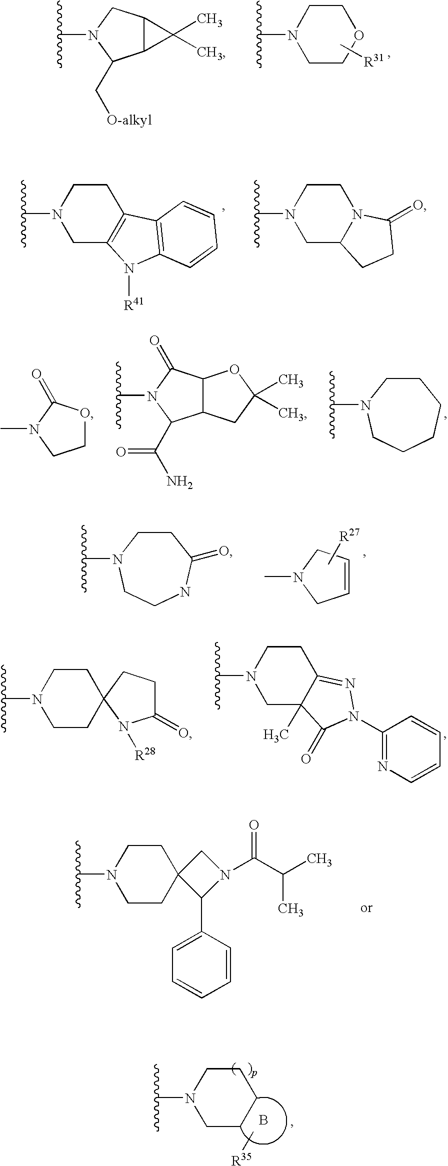

wherein

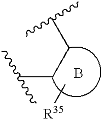

comprises an R³⁵-substituted 5 or 6-membered heteroaryl group fused to the piperidinyl or pyrrolidinyl ring, “p” and “q” are independently selected from 0 and 1, and a dotted line represents an optional double bond.

The '062 publication further provides the following definitions of sub-groups contained within the definitions of R⁷ and R⁸ presented therein and above, which are intended to be incorporated into like substituent definitions herein:
R¹² is 1-3 substituents independently selected from the group consisting of H, alkyl, hydroxy, alkoxy, hydroxyalkyl, alkoxyalkyl, —C(O)Oalkyl, —(CH₂)ₙ—N(R³⁰)—C(O)-cycloalkyl, —(CH₂)ₙ—N(R³)—C(O)alkyl, —(CH₂)ₙ—N(R³⁰)—C(O)Oalkyl, —(CH₂)ₙ—N(R³⁰)—(R²³⁻heteroaryl), —(CH₂)ₙ—N(R³⁰)—C(O)—NR¹⁸R¹⁹, —(CH₂)ₙ—C(O)—NR¹⁸R¹⁹, R¹⁷-phenyl, R³⁵-heteroarylalkyl, R³⁵-heteroaryloxy, —C(O)-heterocycloalkyl, —O—C(O)-heterocycloalkyl, —O—C(O)—NR¹⁸R¹⁹, —NH—SO₂-alkyl, —NH—C(═NR)NH₂, and

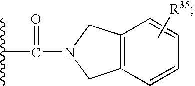

or two R¹² substituents on the same carbon form ═O, ═NOR³⁰ or ═CH₂;
R¹⁴ is 1 or 2 substituents independently selected from the group consisting of H, OH, halo, alkyl, alkoxy, hydroxyalkyl, alkoxyalkyl, —CF₃, CN, R¹⁷-phenyl, (R¹⁷-phenyl)alkyl, —NR¹⁶R¹⁹, alkyl-NR¹⁸R¹⁹, —(CH₂)ₙ—C(O)OH, —(CH₂)ₙ—C(O)Oalkyl, —(CH₂)ₙ—C(O)alkyl, —(CH₂)ₙ—C(O)(R³⁵-phenyl), —(CH₂)ₙ—C(O)(R²³-heteroaryl), —(CH₂)ₙ—C(O)NR¹⁸R¹⁹, —(CH₂)ₙ—C(O)N(R³⁰)—(CH₂)ₙ—(R²³-heteroaryl), —(CH₂)ₙ—N(R³⁰)—C(O)alkyl, —(CH₂)ₙ—N(R³⁰ —C(O)-(fluoroalkyl), —(CH₂)ₙ—N(R³⁰)—C(O)-(cycloalkyl), —(CH₂)ₙ—N(R³⁰)—C(O)(R³⁵-phenyl), —(CH₂)ₙ—N(R³⁰)—C(O)(R²³-heteroaryl), —(CH₂)ₙ—N(R³⁰)C(O)NR¹⁸R¹⁹, —(CH₂)ₙ—N(R³⁰)—C(O)Oalkyl, —(CH₂)ₙ—N(R³⁰)cycloalkyl, —(CH₂)ₙ—N(R³⁰)(R¹⁷-phenyl), —(CH₂)ₙ—N(R³⁰)(R²³-heteroaryl), —(CH₂)ₙ—N(R¹⁸)SO₂alkyl, —(CH₂)ₙ—N(R²⁰)SO₂—(R¹⁷-phenyl), —(CH₂)ₙ—N(R³⁰)SO₂—CF₃, —CH₂S(O)₀₋₂(R³⁵-phenyl), —(CH₂)ₙ—OC(O)N(R³⁰)alkyl, R²³-heteroaryl, (R²³-heteroaryl)alkyl, (R²³-heteroaryl)oxy, (R²³-heteroaryl)amino, —CH(OH)—(R¹⁷-phenyl), —CH(OH)—(R²³-heteroaryl), —C(═NOR³⁰)—(R¹⁷-phenyl), —C(═NOR³⁰)—(R²³-heteroaryl), morpholinyl, thiomorpholinyl,

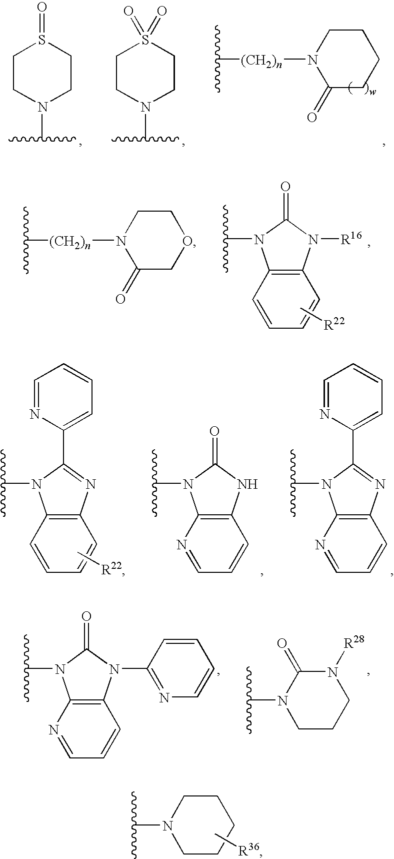

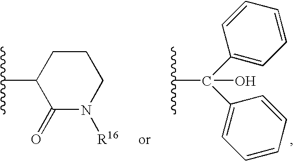

w is 0 or 1, or two R¹⁴ substituents and the carbon to which they are both attached form —C(═NOR³⁰)— or —C(O)—;

each n is independently 0, 1, 2 or 3;
R¹⁵ is H, alkyl, cycloalkyl, (cycloalkyl)alkyl, hydroxyalkyl, alkoxyalkyl, haloalkyl, —C(O)Oalkyl, —C(O)O(R³⁰-cycloalkyl), -alkyl-C(O)O-alkyl, —C(O)O-alkylene-(R³⁵-phenyl), R¹⁷-phenyl, (R¹⁷-phenyl)alkyl, —CH—(R¹⁷-phenyl)₂, R²³-heteroaryl, —(CH₂)ₙ—C(O)NR¹⁸R¹⁹, —SO₂-alkyl, —SO₂-cycloalkyl, —SO₂—CF₃, —SO₂—(R³⁵-phenyl), —SO₂—NR¹⁸R¹⁹, —C(O)alkyl, —C(O)-(fluoroalkyl), —C(O)—C(CH₃)(CF₃)₂, —C(O)—(R¹⁷-phenyl), —C(O)—(R²³-heteroaryl), —C(O)-hydroxyalkyl, —C(O)-alkoxyalkyl, —C(O)—(R³⁹-cycloalkyl), —C(O)-alkylene-(R¹⁷-phenyl), —C(O)-alkylene-(R²³-heteroaryl), —C(O)-alkylene-S—C(O)alkyl, —C(═S)—(R¹⁷-phenyl), hydroxyalkyl substituted by R¹⁷-phenyl, hydroxyalkyl substituted by R²³-heteroaryl, alkoxyalkyl substituted by R¹⁷-phenyl, alkoxyalkyl substituted by R²³-heteroaryl,

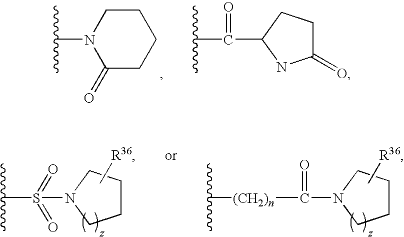

wherein
z is 0, 1 or 2;
R¹⁶ is 1 to 4 substituents independently selected from the group consisting of H, alkyl, R¹⁷-phenyl, (R¹⁷-phenyl)alkyl, (R²³-heteroaryl)alkyl, hydroxyalkyl, alkoxyalkyl and —C(O)Oalkyl, or two R¹⁶ groups and the carbon to which they are both attached form —C(O)—;
R¹⁷ is 1 to 3 substituents independently selected from the group consisting of H, halo, alkyl, cycloalkyl, —OH, hydroxyalkyl, alkoxy, alkoxyalkyl, —CN, —CF₃, —OCF₃, —OCHF₂, —OCH₂F, —C(O)OH, —C(O)Oalkyl, —C(O)O—(R³⁵-phenyl), —C(O)alkyl, —C(O)—(R³⁵-phenyl), —SOalkyl, —SO₂alkyl, —SO₂—CF₃, alkylthio, —NR⁴³R⁴⁴, -alkyl-NR⁴³R⁴⁴, R³⁵-phenyl, R³⁵-phenoxy, R³⁵-heteroaryl, R³⁵-heteroaryloxy, R³⁶-heterocycloalkyl, —C(O)—(R³⁶-heterocycloalkyl), hydroxyalkyl-NH—, —C(O)N(R³⁰)₂, —N(R⁴³)—(R³⁵-cycloalkyl) and —C(═NOR³⁰); or two R¹⁷ substituents on adjacent carbon atoms together form —O—CH₂—O—, —O—(CH₂)₂—O—, —(CH₂)₂—O— or —O—CH₂—O—CH₂—;
R¹⁸ and R¹⁹ are independently selected from the group consisting of H, alkyl, hydroxyalkyl, alkoxyalkyl, haloalkyl, R¹⁷-phenyl, (R¹⁷-phenyl)alkyl, naphthyl and cycloalkyl;
R²⁰ is H, alkyl, or cycloalkyl;
R²² is 1 to 4 substituents independently selected from the group consisting of H, alkyl, hydroxy, alkoxy, halo, —CF₃, —NH₂ and R³⁵-phenyl;
R²² is 1 to 4 substituents independently selected from the group consisting of H, alkyl, hydroxy, alkoxy, halo, —CF₃, —NR¹⁸R¹⁹ CN, —C(O)Oalkyl, —SO₂-alkyl, —NHSO₂-alkyl, R³⁵-phenyl, R³⁵-heteroaryl, morpholinyl, and —(CH₂)ₙ—C(O)—N(R³⁰)₂;
R²⁴ is H, OH or alkoxy; or when the optional double bond is present, R²⁴ and the adjacent carbon atom form the double bond;
R²⁸ is H or R³⁵-phenyl;
R²⁷ is 1 to 3 substituents independently selected from the group consisting of H, halo, OH, alkyl, alkoxy, hydroxyalkyl, alkoxyalkyl, haloalkyl, —CN, —C(O)OH, —C(O)Oalkyl, —C(O)N(R³⁰)(R¹⁸), —C(O)—(R³⁶⁻heterocycloalkyl), R¹⁷-phenyl, (R¹⁷-phenyl)-alkyl, R²³-heteroaryl, (R²³-heteroaryl)alkyl, (R²³-heteroaryl)oxy, (R²³-heteroaryl)amino, NR¹⁸R¹⁹, NR¹⁸R¹⁹-alkyl, —(CH₂)ₙ—N(R³⁰)—C(O)alkyl, —(CH₂)ₙ—N(R³⁰)—C(O)-(fluoroalkyl), —(CH₂)ₙ—N(R³⁰)—C(O)alkoxyalkyl, —(CH₂)ₙ—N(R³⁰)—C(O)(cycloalkyl), —(CH₂)ₙ—N(R³)—(R²³-heteroaryl), —(CH₂)ₙ—N(R³⁰)—C(O)—(R²³-heteroaryl), —(CH₂)ₙ—N(R³⁰)—C(O)O-alkyl, —(CH₂)ₙ—N(R³⁰)—C(O)O—(CF₃-alkyl), —(CH₂)ₙ—N(R³⁰)—C(O)O—(R³⁹⁻cycloalkyl), —(CH₂)ₙ—N(R³⁰)—C(O)O-alkylene-cycloalkyl, —(CH₂)ₙ—N(R³⁰)—C(O)—N(R³⁰)(R²⁰), —(CH₂)ₙ—N(R³⁰)—SO₂-alkyl, —(CH₂)ₙ—N(R³⁰)—SO₂—CF₃, —(CH₂)ₙ—N(R³⁰)—SO₂—N(R³⁰)₂ and

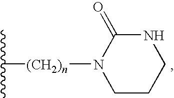

or two R²⁷ groups and the carbon to which they are both attached form —C(═NOR³⁰)— or —C(O)—;
R²⁸ is H, alkyl, R³⁵-benzyl or -alkyl-C(O)O-alkyl;
R²⁹ is alkyl, haloalkyl, —C(O)Oalkyl, —C(O)alkyl, —C(O)CF₃, —C(O)—(R¹²-cycloalkyl), —C(O)—(R¹⁷-phenyl), —C(O)—(R²³-heteroaryl), —C(O)—(R³⁶⁻heterocycloalkyl), —SO₂-alkyl, —SO₂—(R³⁵-phenyl), —C(O)NR¹⁸R¹⁹, R³⁵-phenyl, (R³⁵-phenyl)alkyl or R²³-heteroaryl;
R³⁰ is independently selected from the group consisting of H, alkyl, R³⁵-benzyl and R³⁵-phenyl;
R³¹ is H, R³⁵-benzyl or phenoxyalkyl;
R³³ is H, OH, or alkoxy;
R³⁴ is H, alkyl, hydroxyalkyl, alkoxyalkyl or —C(O)Oalkyl;
R³⁵ is 1 to 3 substituents independently selected from the group consisting of H, halo, alkyl, OH, —CF₃, alkoxy, —CO₂alkyl and —N(R⁴³)(R⁴⁴);
R³⁶ is 1 or 2 substituents independently selected from the group consisting of H, alkyl, R¹⁷-phenyl, —OH, hydroxyalkyl, alkoxyalkyl, —C(O)Oalkyl and —NR¹⁸R¹⁹, or two R³⁶ groups and the carbon to which they are both attached form —C(═NOR³⁰)— or —C(O)—;
R³⁷ and R³⁸ are independently selected from the group consisting of H and alkyl, or R³⁷ and R³⁸ together are —(CH₂)₃— or —(CH₂)₄—, and together with the nitrogen to which they are attached, form a ring;
R³⁹ is H, OH, alkyl, alkoxy, or CF₃;
R⁴⁰ is —OR³⁹ or —NHC(O)alkyl;
R⁴¹ is H or —SO₂alkyl;
R⁴² is —(CH₂)ₙ—(R³⁵-phenyl), —(CH₂)ₙ —(R²³-heteroaryl), —C(O)Oalkyl or —C(O)alkyl;
R⁴³ and R⁴⁴ are independently selected from the group consisting of H and alkyl; and
R⁴⁵ is 1 or 2 substituents independently selected from the group consisting of halo, alkoxyalkyl, —CO₂alkyl, R¹⁷-phenyl, R²³-heteroaryl and cycloalkyl.

As used in the '062 publication, the following definitions are applied to the equivalent terms used herein unless at the point of their use herein the terms are defined otherwise:

“Alkyl” means an aliphatic hydrocarbon group which may be straight or branched and comprising about 1 to about 6 carbon atoms in the chain. Branched means that one or more lower alkyl groups such as methyl, ethyl or propyl, are attached to a linear alkyl chain. Non-limiting examples of suitable alkyl groups include methyl, ethyl, n-propyl, isopropyl, n-butyl, t-butyl and n-pentyl.

“Alkenyl” means an aliphatic hydrocarbon group containing at least one carbon-carbon double bond and which may be straight or branched and comprising about 2 to about 6 carbon atoms in the chain. Branched means that one or more lower alkyl groups such as methyl, ethyl or propyl, are attached to a linear alkenyl chain. Non-limiting examples of suitable alkenyl groups include ethenyl, propenyl, n-butenyl, 3-methylbut-2-enyl and n-pentenyl.

“Alkylene” means a difunctional group obtained by removal of a hydrogen atom from an alkyl group that is defined above. Non-limiting examples of alkylene include methylene (i.e., —CH₂—), ethylene (i.e., —CH₂—CH₂—) and branched chains such as —CH(CH₃)—CH₂—.

“Heteroaryl” means a single ring, bicyclic or benzofused heteroaromatic group of 5 to 10 atoms comprised of 2 to 9 carbon atoms and 1 to 4 heteroatoms independently selected from the group consisting of N, O and S, provided that the rings do not include adjacent oxygen and/or sulfur atoms. N-oxides of the ring nitrogens are also included. Examples of single-ring heteroaryl groups are pyridyl, oxazolyl, isoxazolyl, oxadiazolyl, furanyl, pyrrolyl, thienyl, imidazolyl, pyrazolyl, tetrazolyl, thiazolyl, isothiazolyl, thiadiazolyl, pyrazinyl, pyrimidyl, pyridazinyl and triazolyl. Examples of bicyclic heteroaryl groups are naphthyridyl (e.g., 1,5 or 1,7), imidazopyridyl, pyriclopyrimidinyl and 7-azaindolyl. Examples of benzofused heteroaryl groups are indolyl, quinolyl, isoquinolyl, phthalazinyl, benzothienyl (i.e., thianaphthenyl), benzimidazolyl, benzofuranyl, benzoxazolyl, benzisoxazolyl, benzothiazolyl and benzofurazanyl. All positional isomers are contemplated, e.g., 2-pyridyl, 3-pyridyl and 4-pyridyl. The term R²³-heteroaryl refers to such groups wherein substitutable ring carbon atoms have a substituent as defined above. When the heteroaryl group is a benzofused ring, the substituents can be attached to either or both the phenyl ring portion and the heteroaromatic ring portion, and the heteroaryl group can be attached to the rest of the molecule either through the phenyl ring portion or the heteroaromatic ring portion.

“Cycloalkyl” means a non-aromatic mono- or multicyclic ring system comprising about 3 to about 10 carbon atoms, preferably about 3 to about 6 carbon atoms. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalin, norbornyl, adamantyl and the like. Monocyclic rings are preferred.

“Halo” means fluoro, chloro, bromo, or iodo groups. Preferred are fluoro, chloro or bromo, and more preferred are fluoro and chloro.

“Haloalkyl” means an alkyl as defined above wherein one or more hydrogen atoms on the alkyl is replaced by a halo group defined above; in particular, fluoroalkyl refers to an alkyl chain substituted by one or more fluoro atoms.

“Aminoalkyl” means an alkyl as defined above wherein a hydrogen atom on the alkyl is replaced by an amino (i.e., —NH₂) group.

“Heterocycloalkyl” means a non-aromatic saturated monocyclic or multicyclic ring system comprising about 3 to about 10 ring atoms, preferably about 5 to about 10 ring atoms, in which one or more, preferably 1, 2, 3 or 4, of the atoms in the ring system is independently selected from an element other than carbon, for example nitrogen, oxygen or sulfur, alone or in combination. There are no adjacent oxygen and/or sulfur atoms present in the ring system. Preferred heterocycloalkyls contain 5 to 6 ring atoms. The prefix aza, oxa or thia before the heterocycloalkyl root name means that at least a nitrogen, oxygen or sulfur atom respectively is present as a ring atom. The nitrogen or sulfur atom of the heterocyclyl can be optionally oxidized to the corresponding N-oxide, S-oxide or S-dioxide. Non-limiting examples of suitable monocyclic heterocyclyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like. The heterocycloalkyl group can be attached to the parent moiety through a ring carbon or a ring nitrogen.

“(Heterocycloalkyl)alkyl” means a heterocycloalkyl-alkyl group in which the heterocycloalkyl and alkyl groups are as defined above. The bond to the parent is through the alkyl.

“(Heteroaryl)alkyl” means a heteroaryl-alkyl- group in which the heteroaryl and alkyl are as previously described. Non-limiting examples of suitable heteroarylalkyl groups include pyridylmethyl. 2-(furan-3-yl)ethyl and quinolin-3-ylmethyl. The bond to the parent moiety is through the alkyl.

“(Phenyl)alkyl and “(naphthyl)alkyl similarly mean phenyl-alkyl and naphthyl-alkyl groups wherein the bond to the parent moiety is through the alkyl.

“Hydroxyalkyl” means a HO-alkyl- group in which alkyl is as previously defined. Non-limiting examples of suitable hydroxyalkyl groups include hydroxymethyl and 2-hydroxyethyl. Similarly, “dihydroxyalkyl” refers to a straight or branched alkyl chain substituted by two hydroxy groups.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Alkylthio” means an alkyl-S— group in which the alkyl group is as previously described. Non-limiting examples of suitable alkylthio groups include methylthio, ethylthio and isopropylthio. The bond to the parent moiety is through the sulfur.

“Heteroarylamino” means an heteroaryl-NH— group in which the heteroaryl group is as previously described. Non-limiting examples of suitable heteroarylamino groups include pyrimidinyl-amino and pyrazinyl-amino. The bond to the parent moiety is through the amino nitrogen.

“Heteroaryloxy” means an heteroaryl-O— group in which the heteroaryl group is as previously described. Non-limiting examples of suitable heteroaryloxy groups include pyrimidinyl-O— and pyrazinyl-O—. The bond to the parent moiety is through the ether oxygen.

The term “hydroxyalkyl substituted by CO₂alkyl” means an alkyl chain substituted by a hydroxy group and a CO₂alkyl group. Similarly, terms such as “hydroxyalkyl substituted by R¹⁷-phenyl” means an alkyl chain substituted by a hydroxy group and a R¹⁷-phenyl group; “hydroxyalkyl substituted by R¹⁷-phenyl and alkoxy” means an alkyl group substituted by a hydroxy group, a R¹⁷-phenyl, and an alkoxy group. In each of these substituents and other similar substituents listed in the definitions, the alkyl chains can be branched.

Examples of moieties formed when two adjacent R¹⁷ groups form a ring with the carbons on the phenyl ring to which they are attached are:

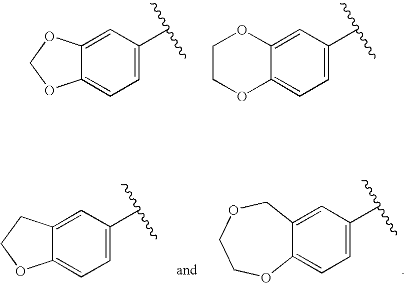

When R⁷ and R⁸ together form

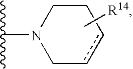

the dotted line indicates an optional double bond as defined above. When the double bond is absent, i.e., when a single bond is present, the one or two R¹⁴ substituents can be attached to the same or different ring carbons. When the double bond is present, only one R¹⁴ substituent can be attached to a carbon that is part of the double bond.

When R⁷ and R⁸ together form

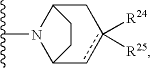

the dotted line indicates an optional double bond as defined above. When the double bond is absent, i.e., when a single bond is present, R²⁴ can be H, OH or alkoxy and R²⁵ can be H or R³⁵-phenyl, but when the double bond is present, R²⁴ forms the double bond with the adjacent carbon and R²⁵ is H or R³⁵-phenyl. That is, the moiety has the structural formula

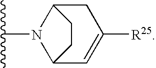

When R⁷ and R⁸ together form

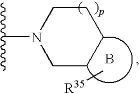

it means that an optionally substituted fused bicyclic ring is formed, wherein the

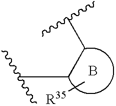

portion comprises an R³⁵-substituted 5 or 6-membered heteroaryl group fused to the piperidinyl ring, Examples are:

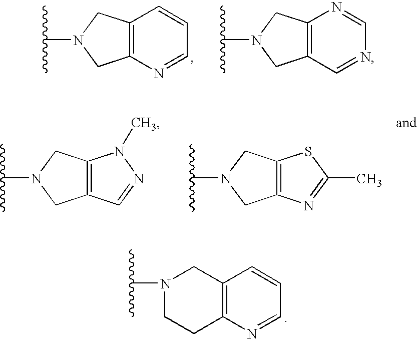

The term “optionally substituted” means optional substitution with the specified groups, radicals or moieties, in available position or positions.

With reference to the number of moieties (e.g., substituents, groups or rings) in a compound, unless otherwise defined, the phrases “one or more” and “at least one” mean that there can be as many moieties as chemically permitted, and the determination of the maximum number of such moieties is well within the knowledge of those skilled in the art.

The wavy line [symbol] as a bond generally indicates a mixture of, or either of, the possible isomers, e.g., containing (R)- and (S)-stereochemistry. For example,

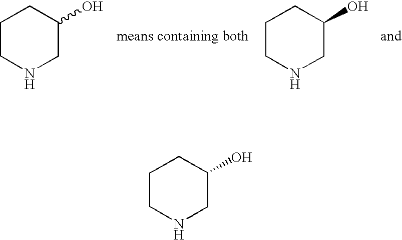

Lines drawn into the ring systems, such as, for example:

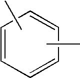

indicate that the indicated line (bond) may be attached to any of the substitutable ring carbon atoms.

As well known in the art, a bond drawn from a particular atom wherein no moiety is depicted at the terminal end of the bond indicates a methyl group bound through that bond to the atom, unless stated otherwise. For example:

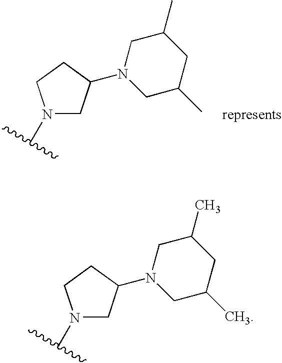

As illustrated herein, any carbon or heteroatom with unsatisfied valences is assumed to have sufficient hydrogen atoms present to satisfy the valences.

The inventors have surprisingly found advantages in preparing the compound of Formula I in accordance with the process shown in Scheme Ia2 in that the individual steps utilize reagents and procedures which are amenable to scaling the process up to provide commercial quantities of the compound of Formula I.

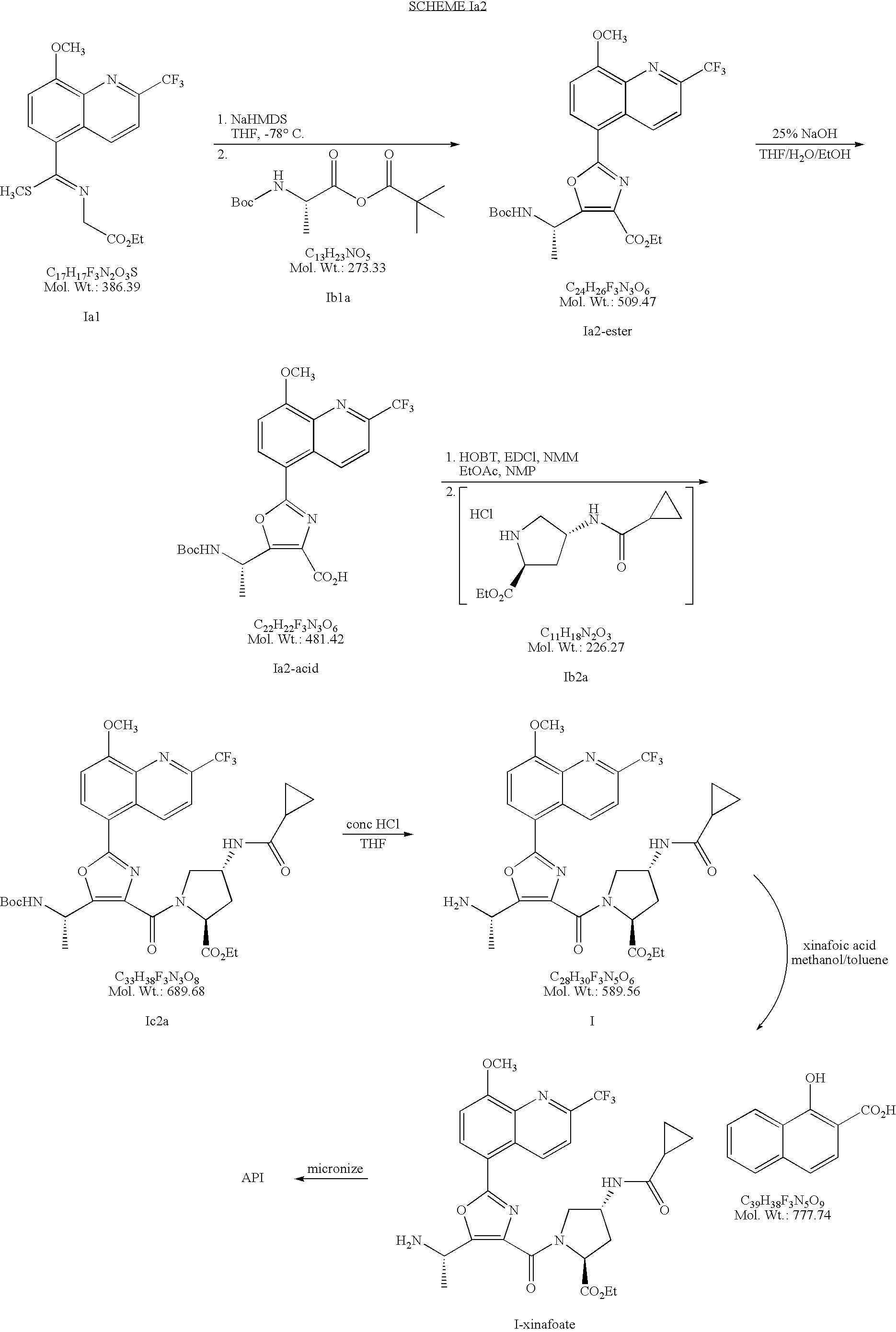

In particular, the inventors have surprisingly found that the formation of trisubstituted oxazole compounds, for example, with reference to Schemes Ia and Ia2, the compound of Formula Ia2-ester, prepared using an acid substituted with an acid activating moiety, for example, an acid anhydride (wherein the acid activating moiety is R—C(O)—O—), for example, the anhydride compound of Formula Ib2a as shown in Scheme Ia2, provides an oxazole product having fewer impurities when impurity levels are compared in the product of a reaction carried out using an acid halide, for example, as shown in Scheme Ia, the formation of the compound of Formula Ib1.

Moreover, the inventors have surprisingly found that the use of a salt compound of Formula Ib2a in reaction with the compound of Formula Ia2-acid, yielding the compound of Formula Ic2a, allows the amine source (salt compound of Formula Ib2a) to be prepared at a time and place remote from the reaction yielding the compound of Formula Ic2a, whereas, reactions utilizing the freebase form of the compound of Formula Ib2a (for example, with reference to Scheme Ia, the compound of Formula Ib2) is not stable to isolation, and therefore must be prepared contemporaneously and locally to its use in a reaction yielding the compound of Formula Ic2a.

Additionally, the inventors have surprisingly found that utilizing an agitated dryer of the type consistent with cGMP practices of the pharmaceutical industry to dry material obtained by precipitating and drying the xinafoate salt of the compound of Formula I can yield a particulate material having acceptable particle size distribution and mean particle size if the following drying protocol is utilized: (a) Tⱼ=50° C., no agitation for 3 hours at 0.1 bar pressure; (b) Tⱼ=80° C., agitation set at 20 rpm for 12 hours at 0.1 bar pressure; and (c) Tⱼ=80° C., agitation set at 60 rpm for 12 hours at 0.1 bar pressure, wherein Tⱼ is the jacket temperature of the rotary dryer used to dry the compound.

With reference to the process illustrated herein by Scheme Ia2, in Step “b”, wherein the compound of Formula (Ia1) is reacted first with an alkali metal amide base and then with an an acid substituted with an acid activating moiety, for example, an acid anhydride, and without wanting to be bound by theory, it is believed that there is great potential for decomposition products if the product of the reaction between the compound of Formula (Ia1) and an alkali metal amide base is not quenched rapidly with an anhydride or equivalent acid substituted by an activating moiety or if in reacting the two, excess amide base is present along with the reaction product. Accordingly, the inventors have surprisingly discovered that by utilizing a process wherein a small aliquot of the alikali metal amide base is introduced stepwise into the reaction mixture, preferably in an amount which is completely consumed in reaction with the compound of Formula Ia1 quickly followed by adding to the reaction mixture an equivalent amount of the anhydride of Formula Ib1a, there is yielded a product which has fewer decomposition and side products than when the reaction is run in bulk or when it is run using the acid-halide instead of the anhydride, for example, the compound of Formula Ib1 shown above in Scheme Ia.

Moreover, the inventors have surprisingly found that a continuous flow system in which a stream comprising the compound of Formula Ia1 is combined in a mixing chamber, for example, a mixing tee, with a stream comprising an alkali metal amide, for example, sodium bis(trimethylsilyl)amide (NaHMDS), and after appropriate reaction period, immediately quenching the mixture in a reaction vessel containing a quenching medium comprising the anhydride of Formula Ib1a, yields a cleaner reaction product, with reduced production of decomposition and side reaction products, and improved utilization of the starting materials compared to the process shown in Scheme Ia. The continuous flow reaction scheme also permits scale up to large scale batches using this reaction chemistry as it eliminates the necessity to interleave aliquots of amide and anhydride in multiple separate additions to the reaction vessel.

In some embodiments of the continuous reaction, it is preferred to carry out the reaction process at a temperature of 0° C. or lower, preferably at a temperature of about [−25° C.] or lower, more preferably at a temperature of less than about [−50° C.], and more preferably at a temperature of about [−75° C.] or lower given the rapid reaction kinetics involved.

It will be appreciated that the advantages offered by the present invention in the preparation of the oxazole of Formula Ia2 can equally be applied to the formation of any of the intermediate oxazoles described in the above-referenced '062 publication. Thus, the reaction can be used to react a compound of the formula:

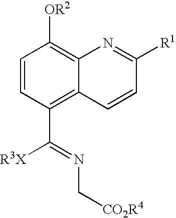

with a compound of formula:

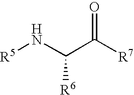

to yield a compound of formula:

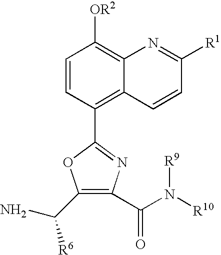

or a salt thereof;
wherein
   R¹ is a haloalkyl;
   R² is alkyl;
   R⁶ is hydrogen, methyl, alkyl of 2 carbons or more, hydroxyalkyl, alkoxyalkyl, mercaptoalkyl, —CH₂F, —CHF₂, —CF₃, —C(O)OH, —C(O)Oalkyl or —C(O)NR⁴³R⁴⁴, where R⁴³ and R⁴⁴ are independently H or alkyl;
   R⁹ is a group defined as R⁷ in published US patent application No. 2006/0106062; and
   R¹⁰ is a group defined as R⁸ in published US patent application No. 2006/0106062;
      said process comprising the following:
a) reacting a compound of the formula:

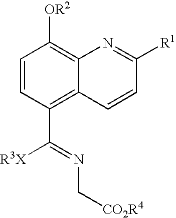

wherein
   R¹ and R² is as defined above;
   R³ are selected from hydrogen and alkyl;
   R⁴ is alkyl; and
   X is selected from oxygen and sulfur;
      with a compound of the formula:

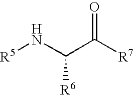

wherein
   R⁵ is an acid sensitive amino protecting group;
   R⁶ is as defined above;
   R⁷ is a halide or an acid activating moiety, for example, an alkylcarbonyloxy moiety, a morpholino moiety, or an imidazole moiety, preferably R⁷ is an alkylcarbonyloxy moiety, in which case the compound is an acid anhydride;
      to yield a compound of the formula:

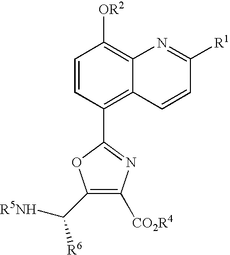

wherein R¹, R², R⁴, R⁵, and R⁶ are as defined above;
b) converting the compound of the formula:

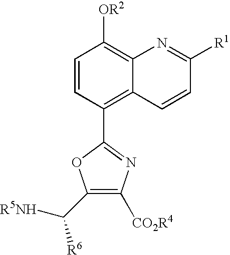

to the compound of the formula:

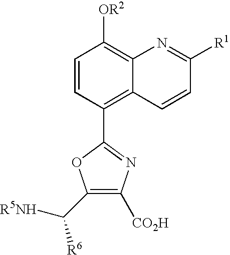

c) reacting the compound of the formula:

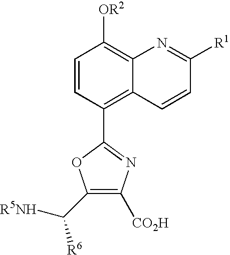

with the compound of the formula:

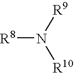

wherein
   R⁸ is hydrogen or alkoxycarbonyl, and
   R⁹ and R¹⁰ are as defined above;
to give the compound of the formula:

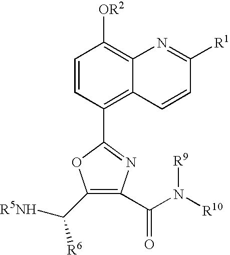

d) removing any protecting groups to yield the compound of the formula:

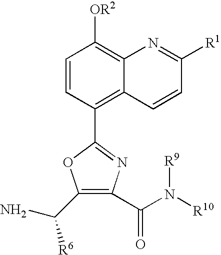

and
e) optionally reacting the compound prepared in step “d” with an acid to form a salt of the formula:

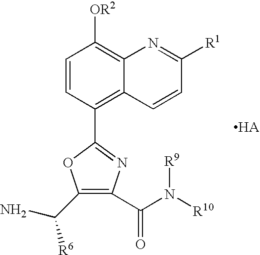

wherein “A” is a pharmaceutically acceptable anion.

In some embodiments of the present invention utilizing the above-described continuous process for preparing oxazole compounds, preparation of the intermediate in step (a) is carried out at a temperature of 0° C. or lower, preferably less than −25° C., more preferably less than −50° C., and even more preferably less than −75° C.

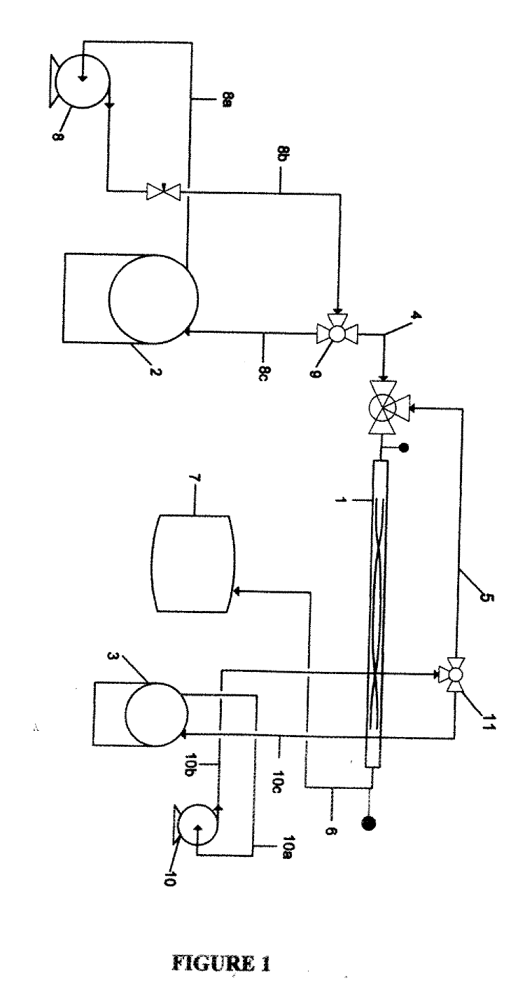

With reference to FIG. I, the reaction described above can be carried out on a continuous basis by using a simple apparatus having a mixing chamber, for example, a simple plumbing tee, with one leg, preferably the run leg, connected (via conduit 4) to a source of the compound of Formula Ia1 (vessel 2) through a 3-way valve. The mixing tee side leg is connected via conduit 5 to a source of the alkali metal amide (vessel 3) through a 3-way valve. As shown in FIG. 1, the continuous reactor apparatus also has two recirculating loops, one comprising pump 8, conduits 8a to 8c, and 3-way valve 9, for recirculating the contents of vessel 2 or pumping the contents of vessel 2 into the mixing chamber, and another comprising pump 10, conduits 10a to 10c and 3-way valve 11, for recirculating the contents of vessel 3 or pumping the contents of vessel 3 into the mixing chamber. The presence of the 3-way valves in each recirculating system permits the contents of the associated vessel (generally reactants in solution) to be circulated within the system between the vessel and the conduit leading to the mixing chamber and thereby equilibrate the liquid residing in the vessel with the reactor to bring the system to the desired temperature prior to the reactants being combined in the mixing chamber.

As shown in FIG. 1, the mixing chamber outlet is directed to static mixer (1) which is sized to an appropriate length to insure that complete reaction occurs. The outlet of the static mixer is directed via conduit (6) to quenching tank (7) which contains the anhydride. Using this type of apparatus, generally the starting materials are cooled to the desired temperature, for example, a temperature of 0° C. or less, preferably less than about [−25° C.], more preferably less than about [−50° C.], and more preferably less than about [−75° C.], and then the valves of the recirculation loop are set to pass the contents into the mixing chamber and thence into the static mixer at substantially the same time. The continuous reaction proceeds in the mixer, and upon completion of the reaction, the resulting intermediate product is quenched with an acid halide or an anhydride, or an acid substituted with an acid activating moiety equivalent to an acid anhydride, in quenching tank 7. A process according to the present invention offers improved control over the reaction chemistry, which allows for improved control of the physical attributes of the final product, for example, increases in product purity and yield. It will be appreciated that other configurations of equipment can be employed which permit pre-reaction of the base and compound of Formula Ia1 prior to quenching, preferably in the anhydride, and not depart from the scope of the invention. It will be appreciated also that the reactor illustrated in FIG. 1 can be scaled appropriately to the volume of material to be processed. Thus, as illustrated, the conduits and vessels shown in FIG. 1 can represent all scales of apparatus from bench top apparatus using flasks, peristaltic pumps, and tubing to industrial scale apparatus employing piping, high capacity pumps, and tanks.

In some embodiments of the invention it is preferred to convert the compound of Formula I produced in the synthesis (for example, the compound of Formula I prepared in accordance with Scheme Ia2, steps “a” to “e”) to an acid salt, for example, a xinafoate salt of a desired crystalline modification. Examples of procedures for preparing such salts are described in the Examples section herein and in U.S. patent application Ser. No. 11/775,383. filed Jul. 10, 2007, which application is incorporated herein by reference, particularly regarding its description of the preparation of the compound of Formula and xinafoate salts thereof. Surprisingly, the inventors have discovered that controlled drying procedures utilized to dry the xinafoate salt yields particles having particularly desirable particle size distribution and average particle size for preparing medicaments to be administered by inhalation, Moreover, the particles thus produced have an acceptable Hauser ratio (bulk density divided by tapped density).

With reference to the process illustrated herein in Scheme Ia2 and described thereafter in process steps “a” through “g”, it has been surprisingly found that after optional step “f” has been carried out, particles having the above-described acceptable Hauser ratio are produced when drying is carried out in an agitated dryer equipped with a jacket and having reduced pressure capabilities, in particular, drying the precipitated salt produced in optional step “f” using an agitated dryer under the following regime: (a) Tⱼ=50° C., no agitation for 3 hours at 0.1 bar pressure; (b) Tⱼ=80° C., agitation set at 20 rpm for 12 hours at 0.1 bar pressure; and (c) Tⱼ=80° C., agitation set at 60 rpm for 12 hours at 0.1 bar pressure, wherein Tⱼ is the jacket temperature of the rotary dryer used to dry the compound.

With reference to the process of Scheme Ia2, and other schemes presented herein for preparation of trisubstituted oxazoles related to the intermediate of Formula Ia2-ester, the use of a base comprising an alkali metal amide has been described. Preferably, the alkali metal amide used is sodium bis(trimethylsilyl)amide (NaHMDS), however, it will be appreciated that other bases in this general class, as well as some other strong bases can be employed also without departing from the scope of the invention.

It will be appreciated that the reagents utilized in the present process can be supplied by any means and not depart from the scope of the invention. In some embodiments it is preferred to supply the anhydride of Formula Ib1a in accordance with step 1 of Example 1, below. In the same manner, for the process of the present invention in accordance with Scheme Ia2, it is preferred to supply the compound of Formula Ib2a by deprotecting the corresponding t-Boc-protected analog in accordance with the process described in Example 1, step 3, part A, however, this compound, or any other amine used in a related procedure reacting the oxazole intermediate with an amine, can employ any means to supply the amine without departing from the scope of the invention.

Upper and lower airway obstructive disease treated by the compound of Formula I include asthma, COPD (chronic obstructive pulmonary disease), chronic bronchitis, cystic fibrosis, allergic rhinitis, non-allergic rhinitis, rhinosinusitis, adult respiratory disease, acute respiratory distress syndrome, respiratory viruses, cough, interstitial pneumonitis, chronic sinusitis, airflow obstruction, airway hyperresponsiveness (i.e., airway hyperreactivity), bronchiectasis, bronchiolitis, bronchiolitis obliterans (i.e., bronchiolitis obliterans syndrome), dyspnea, emphysema, hypercapnea, hyperinflation, hypoxemia, hyperoxia-induced inflammations, pulmonary fibrosis, pulmonary hypertension, small airway disease, wheeze and colds.

Compounds of Formula I are preferably useful in treating asthma, COPD, cough, airflow obstruction, airway hyperresponsiveness (i.e., airway hyperreactivity), bronchiolitis, chronic bronchitis, emphysema, pulmonary fibrosis, pulmonary hypertension, small airway disease, wheeze and allergic rhinitis.

More preferably, compounds of Formula I are useful for treating COPD and asthma.

Other agents for treating an obstructive airway disease (e.g., COPD or asthma) for use in combination with the compound of Formula I are selected from the group consisting of: steroids (e.g. glucocorticoids), 5-lipoxygenase inhibitors, β-2 adrenoceptor agonists, α-adrenergic receptor agonists, muscarinic M1 antagonists, muscarinic M3 antagonists, muscarinic M2 antagonists, LTB4 antagonists, cysteinyl leukotriene antagonists, bronchodilators, PDE4 inhibitors, elastase inhibitors, MMP inhibitors, phospholipase A2 inhibitors, phospholipase D inhibitors, histamine H1 antagonists, histamine H3 antagonists, dopamine agonists, adenosine A2 agonists, NK1, NK2 and NK3 antagonists, GABA-b agonists, nociceptin agonists, expectorants, mucolytic agents, decongestants, mast cell stabilizers, antioxidants, anti-IL-8 anti-bodies, anti-IL-5 antibodies, anti-IgE antibodies, anti-TNF antibodies, IL-10, adhesion molecule inhibitors, growth hormones and other PDE4 inhibitors.

Non-limitative examples of antihistamines that can be used in combination with compounds of Formula I include astemizole, azatadine, azelastine, acrivastine, brompheniramine, certirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, equitazine, mianserin, noberastine, meclizine, norastemizole, picumast, pyrilamine, promethazine, terfenadine, tripelennamine, temelastine, trimeprazine and triprolidine.

Non-limitative examples of histamine H₃ receptor antagonists include: thioperamide, impromidine, burimamide, clobenpropit, impentamine, mifetidine, S-sopromidine, R-sopromidine, SKF-91486, GR-75737, GT-2016, UCL-1199 and clozapine. Other compounds can readily be evaluated to determine activity at H₃ receptors by known methods, including the guinea pig brain membrane assay and the guinea pig neuronal ileum contraction assay, both of which are described in U.S. Pat. No. 5,352,707. Another useful assay utilizes rat brain membranes and is described by West at al., “Identification of Two-H₃-Histamine Receptor Subtypes,” Molecular Pharmacology, Vol. 38, pages 610-613 (1990).

The term “leukotriene inhibitor” includes any agent or compound that inhibits, restrains, retards or otherwise interacts with the action or activity of leukotrienes. Non-limitative examples of leukotriene inhibitors include montelukast and its sodium salt; 1-(((R)-(3-(2-(6,7-difluoro-2-quinolinyl)ethenyl)phenyl)-3-(2-(2-hydroxy-2-propyl)phenyl)thio)methylcyclopropaneacetic acid, and its sodium salt, described in U.S. Pat. No, 5,270,324; 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclo-propaneacetic acid, and its sodium salt, described in U.S. Pat. No. 5,472,964; pranlukast; zafirlukast,; and [2-[[2(4-tert-butyl-2-thiazolyl)-5-benzofuranyl]oxymethyl]phenyl]acetic acid, described in U.S. Pat. No. 5,296,495.

Non-limitative examples of β-adrenergic receptor agonists include: albuterol, bitolterol, isoetharine, mataproterenol, perbuterol, salmeterol, terbutaline, isoproterenol, ephedrine and epinephrine. Non-limitative examples of α-adrenergic receptor agonists include arylalkylamines, (e.g., phenylpropanolamine and pseudephedrine), imidazoles (e.g., naphazoline, oxymetazoline, tetrahydrozoline, and xylometazoline), and cycloalkylamines (e.g., propylhexedrine).

A non-limitative example of a mast cell stabilizer is nedocromil sodium. A non-limitative example of an expectorant is guaifenesin. Non-limitative examples of decongestants are pseudoephedrine, phenylpropanolamine and phenylephrine.

Non-limitative examples of other PDE4 inhibitors include roflumilast, theophylline, rolipram, piclamist, cilomilast and CDP-840. Examples of steroids include prednisolone, fluticasone, triamcinolone, beclomethasone, mometasone, budisamide, betamethasone, dexamethasone, prednisone, flunisolide and cortisone.

Non-limitative examples of NK₁, NK₂ and NK₃ tachykinin receptor antagonists include CP-99,994 and SR 48968. Non-limitative examples of muscarinic antagonists include ipratropium bromide and tiatropium bromide.

Non-limitative examples of GABA_{B} agonists include baclofen and 3-aminopropyl-phosphinic acid. Dopamine agonists include quinpirole, ropinirole, pramipexole, pergolide and bromocriptine.

“5-lipoxygenase inhibitors” include any agent or compound that inhibits, restrains, retards or otherwise interacts with the enzymatic action of 5-lipoxygenase. Non-limitative examples of 5-lipoxygenase inhibitors include zileuton, docebenone, piripost, ICI-D2318, and ABT 761.

The following examples are illustrative of certain embodiments of the present invention. They are not intended to limit the invention to the specific embodiments shown therein.

Below, Scheme 1 and Example 1 exemplify the production of a compound of Formula I according to one embodiment of the present invention, wherein an anhydride is used in Step 1 to produce an intermediate product compound of Formula Ia2-ester (labeled in Scheme 1 as the compound of Formula 2). Scheme 2 and Example 2 exemplify the continuous reaction process for the production of an intermediate ester compound having a similar structure to that of the compound of Formula Ia2-ester, but employing a 9H fluoren-9yl-methoxycarbonyl protecting group (labeled in Scheme 2 as the compound of Formula 8). Scheme 3 and Example 3 detail the continuous reaction for the production of the intermediate of step 1. In Example 1 and elsewhere in the application, Et means ethyl, Me means methyl, LDA is Lithium diisopropylamide, THF is tetrahydrofuran, DMF is N,N-dimethylformamide, t-BOC and BOC mean t-butoxycarbonyl, RT is room temperature, HATU is N-[(dimethylamino)-1H-1,2,3-triazolo[4,5-b]pyridin-1-ylmethylene]-N-methylmethanaminium hexafluorophosphate N-oxide, KF is Karl Fisher titration to determine water content, DMSO is dimethyl sulfoxide, NaMDS is sodium bis(trimethylsilyl)amide, LiHMDS is lithium bis(trimethylsilyl)amide, NMR is nuclear magnetic resonance, HPLC is high performance liquid chromatography, TEA is triethylamine, HOBT is hydroxybenztriazole, EDCI HCl is 1-ethyl-3-[(3-dimethylamino)propyl]-carbiodimide hydrochloride, and NMP is N-methylpurrolidinone.

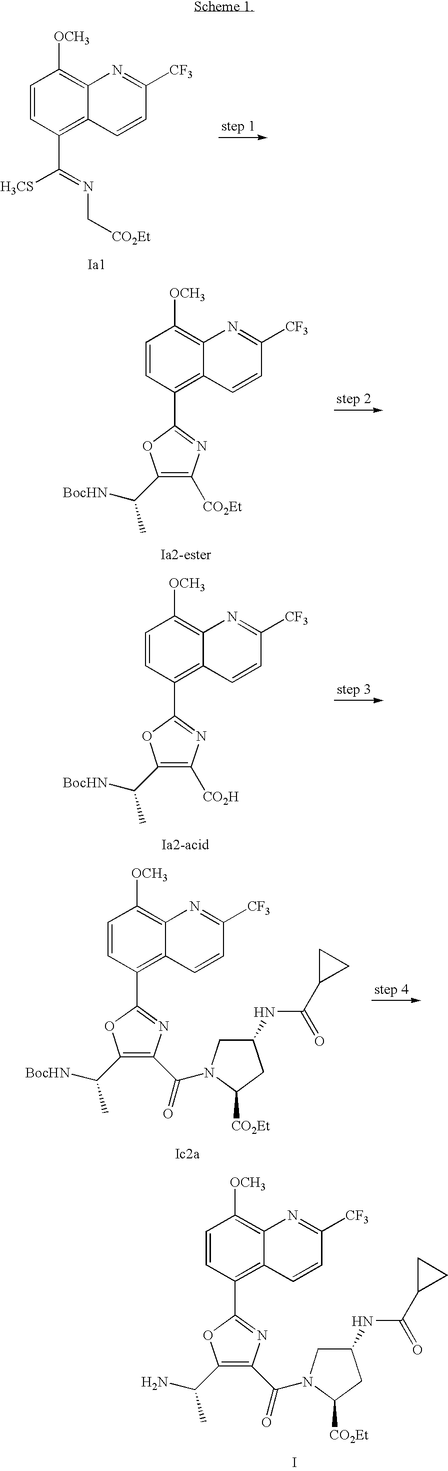

### Example 1

### Step 1:

Into a 50 L Hastelloy reactor equipped with a thermocouple, N₂ inlet and feed tank was charged 8.8 kg (46.5 moles, 2 eq) of (S)-2-tert-butoxycarbonylamino-propionic acid and 90 liters dry tetrahydrofuran (THF, KF<0.05%). The reaction mixture was stirred until all materials were dissolved. Into the reaction mixture was slowly charged 8.5 kg (46.9 moles, 2 eq) of dicyclohexylamine over about 30 minutes maintaining the temperature of the reaction mixture between [−5]° C. and [−5]° C. The batch was agitated for 15 minutes while maintaining the temperature of the reaction mixture between [−5]° C. and [+5]° C. At the end of the agitation period, 5.7 kg (47.3 moles, 2 eq) of trimethylacetylchloride was charged into the reaction mixture over 30 minutes while maintaining the reaction mixture at a temperature between [−5]° C. and [+5]° C. After the addition of trimethylacetylchloride the reaction mixture was agitated for 3 hours maintaining the temperature between [−5]° C. and [+5]° C. At the end of the agitation period, 27 liters of heptane, followed by 4.5 kg of celite was charged into the reaction mixture. The reaction mixture was filterd under N₂ and the filter cake thus obtained was washed with 30% v/v THF in heptane. The filtrate and washes were combined and concentrated by distillation under vacuum to a batch volume of about 36 liters. Into the concentrated reaction mixture was charged 27 liters of THF, and the temperature of the reaction mixture was adjust and maintained at a temperature of from 20° C. to 30° C. The reaction mixture was sampled and the moisture content determined by Kari Fisher titration (KF) to be less than about 0.06 ppm. Thus obtained, this THF solution containing mixed anhydride was used in the next step without further purification.

Into a 50 gallon glass lined reactor equipped with a thermocouple, N₂ inlet and feed tank was charged 9.0 kg (23.3 moles, 1 eq) of the compound (Ia1) and 126 liters dry tetrahydrofuran (THF, KF<0.05%). The mixture was agitated until the solids dissolved. The reaction mixture was concentrated at 1 atmosphere to a batch volume of about 81 liters. The temperature of the reaction mixture was adjusted and maintained between [−60]° C. and [−70]° C. Into the reaction mixture was charged 2.70 Kg of NaHMDS as a 2M solution in THF, (5.9 moles, 0.25 eq) over about 15 minutes maintaining the temperature of the reaction mixture from [−60]° C. to [−70]° C., and the mixture was agitated for 5 minutes while maintaining the temperature. At the end of the agitation period, the mixed anhydride solution in THF prepared in Step 1 (0.83 kg active, 3.2 moles, 0.14 eq) was charged into the reaction mixture over about 15 minutes while maintaining the temperature of the reaction mixture from [−60]° C. to [−70]° C., and following addition, the reaction mixture was agitated for an additional 10 minutes while maintaining the temperature of the reaction mixture. The sequence of two charges (NaHMDS 2M in THF) and the mixed anhydride was repeated seven (7) more times for a total of eight (8) sets of charges, until the conversion of the compound of Formula Ia1 is ≧70%. Charging of NaHMDS (2M in THF) followed by the mixed anhydride in the same ratio based on the amount of starting material remaining is continued until the conversion is ≧94%, then, over a 15 minute period an aqueous solution of 13.5 kg KH₂PO₄ dissolved in 90 liters H₂O is added to the reaction mixture while maintaining the temperature of the reaction mixture below 30° C. At the end of the addition period, 59 liters of ethyl acetate is added and the mixture is agitated for 15 minutes, then the layers are allowed to settle. The aqueous layer is extracted with 45 liters ethyl acetate and the ethyl acetate is combined with the organic layer. The combined organic layers are washed two times with 32 liters 10% aqueous w/v NaCl and concentrated at 1 atmosphere to a volume of about 45 liters. Into the concentrate is charged 90 liters methyltertbutylether (MTBE) and the mixture is concentrated at 1 atmosphere to a batch volume of about 54 liters. Into the concentrate is charged 45 liters of methyltertbutylether while maintaining the temperature between 55° C. and 65° C. followed by 108 liters of heptane while maintaining the temperature between 55° C. and 65° C. The temperature of the reaction mixture was adjusted and maintained at a temperature of from 45° C. to 55° C. and agitate for about 30 minutes. At the end of the agitation period, the temperature of the reaction mixture was adjusted over 1 hour to a temperature of from [5]° C. to [+5]° C., and when adjusted within the range, the mixture was agitated for 30 minutes additional while maintaining the temperature within that range. At the end of the agitation period the reaction mixture was filtered and the filter cake thus obtained was washed with 33% v/v methyltertbutylether in heptane. The solids thus obtained were dried in a vacuum oven for at least 12 hours maintaining the temperature of the oven from 45° C. to 55° C., affording 8.4 kg (72.2%) of (Ia2-ester) as a solid with an ee of >99.0%.

¹H NMR (400 MHz, CDCl₃); 9.89 (1H, d); 8.56 (1H, d); 7.94 (1H, d); 7.22 (1H, d); 5.91 (1H, s,b); 5.58 (1H, s, b); 4.47 (2H, q); 4.43 (3H, s); 3.75 (2H, t); 1.47 (9H, s); 1.19 (9H, s).

### Step 2;

The compound of Formula (Ia2-ester) prepared in Step 1 (20 g, 39.3 mmol, 1 eq) was charged into a 500 mL three-neck round bottom flask fitted with a mechanical stirrer, an additional funnel and a thermocouple followed by 60 ml of THF, 20 mL of EtOH, and 100 mL of water. Into the reaction mixture was placed 8 mL of 25% sodium hydroxide solution and the mixture was agitated at 40° C. for 4 hours. The reaction mixture was monitored by HPLC assay until the ester was consumed, then the reaction mixture was charged with 100 ml of water and heated to 50° C. When the reaction mixture reached 50° C., and while maintaining the reaction mixture at 50° C., to the reaction mixture was charged over 30 minutes 30 ml 1N HCl. The reaction mixture was stirred while maintaining 50° C. for an additional 30 minutes before charging another 24 ml 1N HCl solution over 30 minutes followed by 60 ml of water over 30 minutes at 50° C., then the mixture was cooled to room temperature over 1 hour providing a slurry. The solids in the mixture were collected by suction filtration and the wet cake thus obtained was washed with 40 ml of a mixture of ethanol and water (1/5, v/v). The washed solids were dried under vacuum at 60° C. for 12 h affording 16.8 g (90%) of compound of Formula (IA2-acid) as an off white solid.

¹H NMR (400 MHz, _{d6}-DMSO): 9.97 (1H, d), 8.42 (1H, d), 8.20 (1H, d), 7.48 (1H, d), 5.40 (1H, m), 4.07 (3H, s), 1.45 (3H, d), 1.30 (9H, s)

### Step 3:

Part A

In a vessel, 60 g of (2R,4S)-4(cyclopropanecarbonyl-amino)-pyrrolidine-1,2-dicarboxylic acid-1-tert-butyl ester 2-ethyl ester (BP) (184 mmol, 1 eq) was dissolved in 1.2 L of EtOAc, and a sample of the solution was retained for use as an HPLC standard representing 100% of (BP). The solution was cooled and maintained at a temperature from 20° C. to 35° C. while charging the solution with 36 g of HCl(g) (980 mmol, 5.3 eq). From the reaction mixture thus formed, an HCl salt product precipitated as reaction proceeded. At end of charging the HCl into the reaction mixture, the reaction mixture was maintained at a temperature of from 20° C. to 30° C. and agitated for 1 h, then analyzed by HPLC against the 100% sample taken to determine if the reaction was complete. Agitation was continued until, as determined by HPLC, the amount of (BP) relative to the 100% standard was ≦0.5% in raw area. When the reaction was complete a vacuum was applied to the vessel and the reaction mixture was concentrated to 600 mL volume, forming a thick slurry, by distilling off volatiles under vacuum while maintaining the reaction mixture at a temperature of from 35° C. to 45° C. After the reaction mixture was concentrated, 280 mL of NMP was added to the reaction mixture and it was again concentrated under vacuum (maintaining the reaction mixture at a temperature of from 35° C. to 45° C.) to a volume of about 560 mL, forming a clear solution of the compound of Formula Ib2a (see Scheme Ia2) which was used directly in the coupling step described in Part B.

Part B:

Into a 1 L 3-neck RB flask was dissolve the compound of Formula (IA2-acid) (80 g, 166 mmol, 1 eq), HOBT.H₂O (28 g, 182 mmol, 1.1 eq) and EDCI.HCl (48 g, 250 mmol, 1.4 eq) in NMP (320 mL) and EtOAc (320 mL) with stirring, which was continued for 40 minutes while maintaining the mixture at 25° C. At the end of the initial stirring period, the solution of the compound of Formula (Ib2a) prepared in part A was added to the reaction mixture with continued stirring. At the end of a 10 minute stirring period, N-methyl morpholine (80 mL, 724 mmol, 4.4 eq) was added to the reaction mixture (stirred) at a rate that maintained the temperature of the reaction mixture below 35° C. The completion of the reaction was monitored by sampling aliquots which were analyzed by HPLC. Once the reaction has been judged complete, 320 mL of EtOAc and 800 mL of water was added to the reaction mixture. The resultant mixture was stirred for 15 min after the addition and the layers were separated. The organic layer was washed with a 400 mL aliquot of 1M HCl followed by a 400 mL aliquot of aqueous 10% K₂CO₃ and then a 400 mL aliquot of water. Thus obtained, the reaction mixture was concentrated to ˜160 mL and 800 mL of acetone was added. After acetone addition, the reaction mixture was again concentrated to a volume of 240 mL under reduced pressure while maintaining the reaction mixture at a temperature of from 40° C. to 50° C. Thus obtained the reaction mixture was diluted with another 800 mL of acetone and the mixture again concentrated to ˜240 mL under reduced pressure while maintaining the temperature of the reaction mixture from 40° C. to 50° C. While maintaining the batch temperature at 40° C., 800 mL of heptanes were slowly added to the reaction mixture, resulting in the formation of a precipitate. The precipitated solids were collected by filtration and dried under vacuum at 50° C. for 12 h to afford (103 g, 90%) of (Ic2a) as an off white solid.

NMR (400 MHz. _{d6}-DMSO): 9.55, 9.03, 8.18, 7.90, 7.77, 7.66, 7.10, 7.04, 6.70, 6.66, 6.10, 5.76, 5.36, 4.91, 4.80, 4.4-3.5, 2.58, 2.30, 1.82. 1.56, 1.47, 1.31, 1.07, 1.001.84, 0.74. Note: due to the presence of rotomers, the observed peaks are listed as observed only.

Step 4:

Compound (Ic2a) (20 g, 29 mmol, 1 eq) was charged to a flask then dissolved with THF (60 ml) and the solution was cooled to 0-10° C. Concentrated HCl (20 ml) was added slowly, at a rate permitting the reaction mixture to be maintained at a temperature of from 0° C. to 20° C. When the HCl had been charged, the solution was warmed and maintained at a temperature of from 20° C. to 30° C. and agitated for about 4 h at which time the reaction was determined to be complete by HPLC analysis. The reaction mixture was diluted with 2-Me-THF (120 ml) and THF (40 ml) and the reaction was quenched with 20% K₂CO₃ (110 ml), yielding pH of 8-8.5. After adjusting pH, 80 ml of water was added and the reaction mixture and the mixture was heated to 30° C. to achieve a clean phase split. The batch was settled for about 15 min, the lower aqueous layer separated, and the organic layer was washed with water (80 ml). The organic phase was diluted with 2-Me-THF (200 ml) and then concentrated under reflux at atmospheric pressure to about 100 ml. A solid product was observed at this volume. The mixture was then cooled to a temperature of from 0° C. to 10° C. and filtered. The wet cake thus obtained was washed 2 times with 2-Me-THF (40 ml each time). Following washing the wet cake was dried for at least 12 h at 60° C. under vacuum affording 13.50 g (79%) of the compound of Formula (I) as a white solid,

¹H NMR (spectrum indicates rotomers, only chemical shift is reported, not integration or peak multiplicity; 400 MHz, _{d6}-DMSO) δ 9.82, 9.62, 8.51, 8.38, 8.07, 7.45, 5.46, 4.69, 4.57, 4.33, 4.15, 4.08, 3.99, 3.83, 2.39, 2.26, 2.16, 1,56, 1.44, 1.22, 0.82, 0.69; MSES+m/z (relative intensity) 590 (M+H).

### Example 2

### Production of the intermediate (compound 8) of step 1 using acid halide

The following example references Scheme 2 when identifying compounds by compound number.

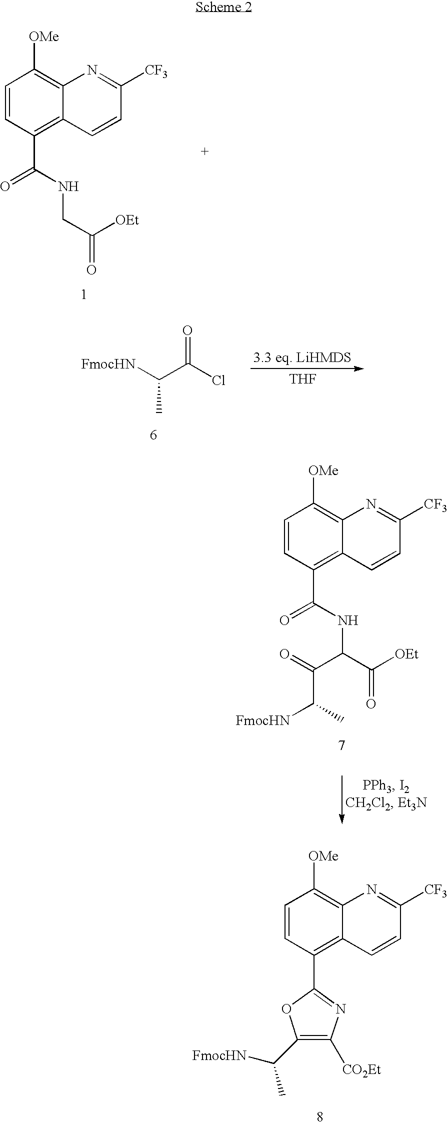

Into a 250 mL flask fitted with magnetic stirring bar and N₂ inlet was charged 7.0 g Fmoc-Ala-OH (22.5 mmol) and 140 mL dichloromethane. The solids were suspended by agitation and 6.8 mL oxalyl chloride was charged. When gas evolution slowed, a catalytic amount of DMF (0.30 mL, 3.9 mmol, 0.17 eq) was charged into the mixture. The reaction was allowed to stir for 3 hours at room temperature, after which solvent was removed in vacuo, and the residue was redissolved in dichloromethane and heptanes trice and rotovapped to a yellow solid. The product 6 was held under vacuum until use.

### Synthesis of Compound 7

A solution of 15 g of 1 in 420 mL THF was charged to CO₂/acetone cooled Flask No. 1 fitted with magnetic stirring, N₂ inlet, and Flask No. 1 was incorporated into one of the recirculating loops of the continuous reactor illustrated in FIG. 1 (see Vessel 2 in FIG. 1). An alkali metal amide base (139 mL 1.0 M LiHMDS) was charged to Flask No. 2 at room temperature, which flask was fitted with magnetic stirring, N₂ inlet, and Flask No. 2 was incorporated into the second of the recirculating loops of the continuous reactor illustrated in FIG. 1 (see Vessel 3 in FIG. 1). The recirculating loops were operated using the contents of the first and second flasks until the system was at thermal equilibrium with the dry ice/acetone baths cooling Flask No. 1, then the two reactor loops were opened at such a rate that compound 1 and the base (NaHMDS) were slowly combined in a roughly 1:3.3 molar ratio in the mixing chamber of the reactor (see the T-junction appended to Static Mixer 1 of FIG. 1). This reaction stream was passed through static mixers, and then immediately quenched into a CO₂/acetone cooled receiving flask fitted with N₂ inlet and magnetic stirrer, containing a solution of 14.4 g compound 6 in 200 mL THF (see vessel 7 in FIG. 1). After all reagents in flasks 1 and 2 had passed into the receiving flask, the mixture in the receiving flask was allowed to stir for an additional 15 minutes before it was quenched with 200 mL of a 15.5% w/v NaH₂PO₄ solution. The mixture was transferred to a separatory funnel, and the aqueous layer extracted with 200 mL ethyl acetate. The organics were combined and solvent removed in vacuo to afford crude 7 as an oil.

### Synthesis of Compound 8

Triphenyl phosphine (0.20 g, 0.76 mmol, 2 eq) and I₂ (0.19 g, 0.76 mmol, 2 eq) were charged to a 100 mL flask fitted with N₂ inlet, magnetic stirring bar, and thermocouple, 40 mL CH₂Cl₂ was charged to the flask, followed by 0.21 mL TEA (1.54 mmol, 4 eq). Once all solids had completely dissolved, a solution of 7 in 12 mL CH₂Cl₂ was charged to the flask. The solution was allowed to stir for 12 hours. The mixture was transferred to a separatory funnel and washed twice with 50 mL 10% w/v sodium bisulfite solution, followed by 25 mL H₂O. The organic layer was concentrated in vacuo to afford 8 as a white solid.

### Example 3

### Continuous reaction for the provision the compound of Formula Ia2-ester) using an anhydride

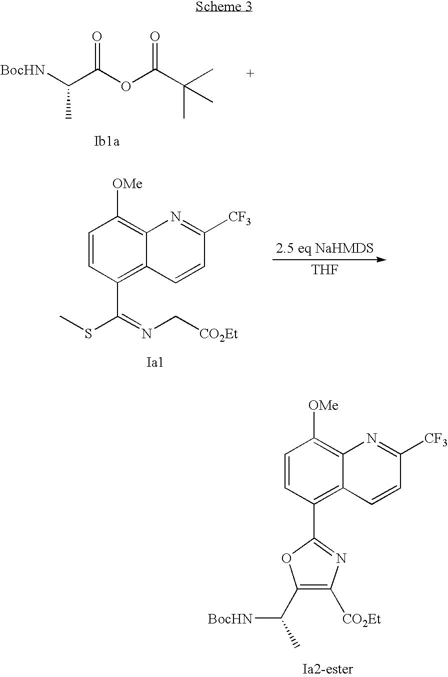

### Synthesis of Compound Ib1a

Into a 500 mL 3-neck round bottom flask fitted with a magnetic stirrer and N₂ inlet was charged 15 g (79.3 mmol, 1 eq) Boc-Ala-OH followed by 180 mL of THF, with stirring, until the material was dissolved. The batch was cooled to 0° C. and 14.4 g of dicyclohexylamine (79.3 mmol, 1 eq) was slowly added at a rate which maintained the temperature of the reaction mixture below 5° C. After the addition was complete, the batch was stirred for 15 min, and then 9.75 g of pivaloyl chloride (80.9 mmol, 1.02 eq) was added to the reaction mixture, with continued stirring while maintaining the mixture at a temperature below 5° C., followed by an additional 3 hours of stirring while maintaining the reaction mixture at a temperature of 0° C. At the end of the stirring period, to the slurry thus formed was added 75 mL of heptanes and 15 g of Celite. The mixture was filtered and the filter cake (celite) was washed with a 100 mL aliquot of a mixture of THF/heptanes (1:2) and the was and filtrates were combined then concentrated in vacuo to afford the compound of Formula (Ib1a) as an oil, used as provided in the Synthesis of the compound of Formula (Ia2-ester).

### Synthesis of the Compound of Formula (Ia2-ester)

A solution of 15 g of the compound of Formula (Ib1a) in 196 ml. THF was charged into a CO₂/acetone cooled Flask no. 2 fitted with magnetic stirring, and N₂ inlet. Flask no. 2 was incorporated into one recirculating loop of a continuous reactor configured as illustrated in FIG. 1 (see Vessel 2 in FIG. 1). Into a CO₂/acetone cooled Flask No. 3 fitted with magnetic stirring and an N₂ inlet was charged 49.0 mL of 2.0 M NaHMDS in THF and Flask No. 3 was incorporated into the second recirculating loop of a continuous reactor configured as illustrated in FIG. 1 (see Vessel 3, FIG. 1). Once the system was at thermal equilibrium with the contents of Flask nos. 2 and 3 recirculating at acetone/dry ice slurry temperature, the two reactor loops were directed to the mixing tee and combined at such a rate that the compound of Formula (Ib1a) and the solution of NaHMDS were slowly combined in a roughly 1:4 molar ratio in the T-junction supplying static mixer (1) of the continuous reactor illustrated in FIG. 1. The combined streams were passed through the static mixer portion of the apparatus and then conducted into a receiving flask fitted with N₂ inlet and magnetic stirrer and containing a solution of 15.9 g the compound of Formula (Ib1a) prepared in the first step dissolved in 154 mL THF cooled to [−20]° C., where the reaction mixture was immediately quenched. After all of the reagents had been passed through the reactor and into the receiving flask, the mixture was allowed to stir in the receiving flask for an additional 15 minutes before adding 150 mL of a 15.5% w/v NaH₂PO₄ solution to the receiving flask, followed by 60 mL ethyl acetate. The mixture in the receiving flask was stirred for an additional 30 min, then transferred to a separatory funnel. The separated aqueous layer thus obtained was extracted with two 50 mL aliquots of ethyl acetate. The organics were collected and combined and the volatile organics were removed in vacuo affording the compound of Formula (Ia2-ester) as a crude product.

Other than as shown in the operating examples or as otherwise indicated, all numbers used in the specification and claims expressing quantities of ingredients, reaction conditions, and so forth, are understood as being modified in all instances by the term “about.” The above description is not intended to detail all modifications and variations of the invention. It will be appreciated by those skilled in the art that changes can be made to the embodiments described above without departing from the inventive concept. It is understood, therefore, that the invention is not limited to the particular embodiments described above, but is intended to cover modifications that are within the spirit and scope of the invention, as defined by the language of the following claims.

1. A process for providing the compound of Formula I, 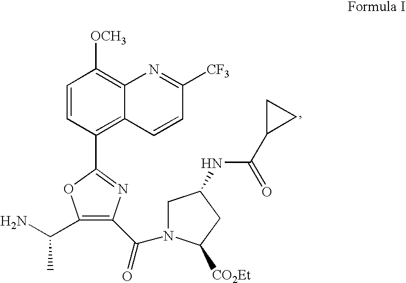
the process comprising:
(a) providing the anhydride of Formula Ib1a, 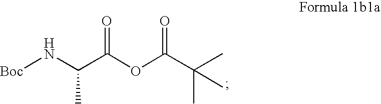
(b) reacting the compound of the Formula Ia1, 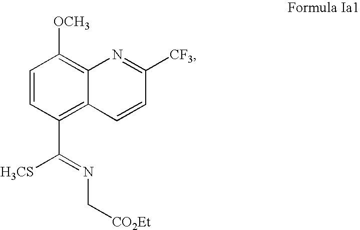
with an alkali metal amide base and reacting that product with the anhydride provided in Step “a” to provide the compound of Formula Ia2-ester, 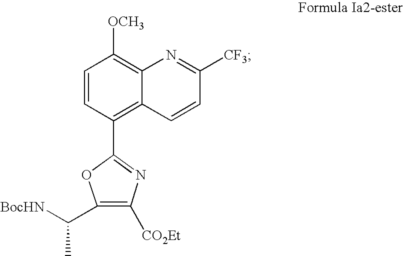
(c) converting the compound of Formula Ia2-ester to the acid of Formula Ia2-acid by treatment with aqueous base; 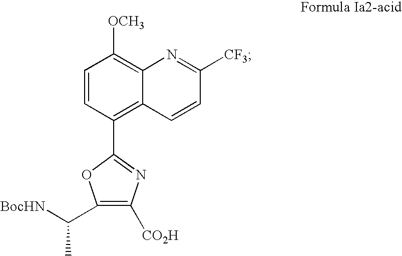
(d) reacting the compound of Formula Ia2-acid with the amino salt compound of the Formula Ib2a, 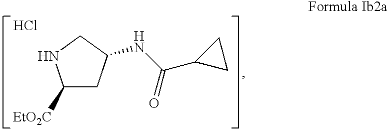
to form the compound of Formula Ic2a, 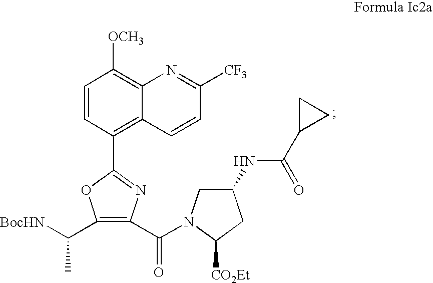
(e) deprotecting the compound of Formula Ic2a formed in Step “d” to form the compound of Formula I;
(f) optionally precipitating the xinafoate salt of the compound of Formula I by treatment with xinafoic acid to provide a compound of the Formula I-xinafoate, 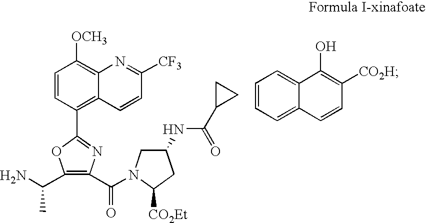
(g) optionally, when optional step “f” has been carried out, drying the precipitated salt in an agitated dryer under the following regime: (a) Tⱼ=50° C., no agitation for 3 hours at 0.1 bar pressure; (b) Tⱼ=80° C., agitation set at 20 rpm for 12 hours at 0.1 bar pressure; and (c) Tⱼ=80° C., agitation set at 60 rpm for 12 hours at 0.1 bar pressure, wherein Tⱼ is the jacket temperature of the rotary dryer used to dry the compound; and
(h) optionally micronizing the compound of Formula I-xinafoate to provide an active pharmaceutical ingredient comprising the compound of Formula I.

2. The process of claim 1, wherein the alkali metal amide base is sodium bis(trimethylsilyl)amide (NaHMDS).

3. The process of claim 2 wherein, the aqueous base used in step “c” is 25% sodium hydroxide and hydrolysis is carried out in the presence of ethanol.

4. The process of claim 2 wherein the anhydride of Formula Ib1a in step “a” is provided by a process comprising reacting the compound of Formula Ib1b (N-Boc-L-alanine), 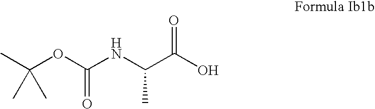
with trimethylacetylchloride in the presence of dicyclohexylamine.

5. The process of claim 2 wherein, step “b” is carried out by placing the compound of Formula Ia1 in a reaction mixture, adding an aliquot of an alkali metal amide base in an amount which is less than required to react with all of the compound of Formula Ia1 present, then adding an equivalent amount of the anhydride compound of Formula Ib1a, and repeating the addition of amide followed by anhydride until substantially all of the compound of Formula Ia1 has been reacted.

6. The process of claim 5 wherein the amide is added in from about 5 to about 10 equal aliquots.

7. The process of claim 2 wherein, step “b” is carried out by combining a first continuous stream of a reaction solvent containing the compound of Formula Ia1 with a second continuous stream of a reaction solvent containing the amide, in a mixing chamber, blending the combined streams in a static mixer, and quenching the mixed streams in a quenching vessel containing the anhydride compound of Formula Ib1a.

8. The process of claim 7 wherein said first and second streams are combined and maintained at a temperature of less than about 0° C. until quenched.

9. The process of claim 7 wherein said first and second streams are combined and maintained at a temperature of less than about [−25° C.] until quenched.

10. The process of claim 7 wherein said first and second streams are combined and maintained at a temperature of less than about [−50° C.] until quenched.

11. The process of claim 7 wherein said first and second streams are combined and maintained at a temperature of less than about [−75° C.] until quenched.

12. The process of claim 2 wherein the compound of Formula Ib2a is provided by reacting the compound of Formula Ib2a1 with gaseous HCl in the presence of ethyl-acetate,

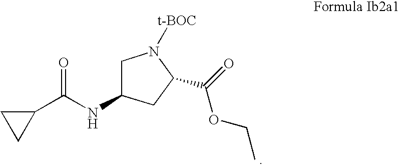

13. A process for preparing an oxazole compound of Formula ID 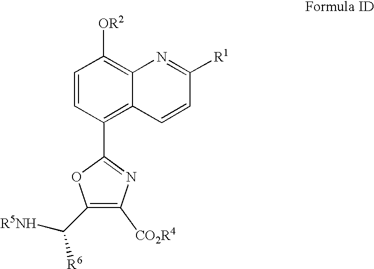
wherein
R¹ is a haloalkyl:
R², R⁴ are alkyl;
R⁵ is an acid labile amino protecting group; and
R⁶ is hydrogen, methyl, alkyl of 2 carbons or more, hydroxyalkyl, alkoxyalkyl, mercaptoalkyl, —CH₂F, —CHF₂, —CF₃, —C(O)OH, —C(O)Oalkyl or —C(O)NR⁴³R⁴⁴, wherein R⁴³ and R⁴⁴ are independently H or alkyl;
the process comprising:
(a) providing a stream comprising the compound of Formula IDa, 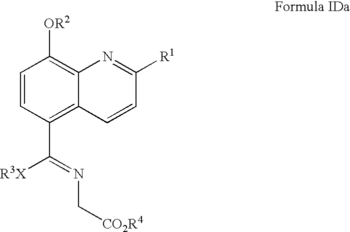
wherein
R¹, R² and R⁴ are defined above;
R³ is hydrogen or alkyl; and
X is oxygen or sulfur;
(b) reacting the stream provided in Step “a” with an alkali metal amide base: and
(c) quenching the reaction mixture from step “b” in a quenching medium comprising a compound of Formula IDb, 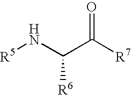
wherein
R⁵ and R⁵ are as defined above; and
R⁷ represents an acid activating moiety.

14. The process of claim 13 wherein R⁷ is an alkylcarbonyloxy moiety.

15. The process of claim 13 wherein R⁷ is a halogen, a morpholino moiety of the formula: 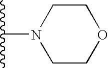
or a derivative thereof, or an imidazole moiety of the formula: 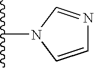
or a derivative thereof.

16. The process of claim 13 wherein the process is carried out at a temperature of less than about 0° C.

17. The process of claim 13 wherein the process is carried out at a temperature of less than about [−25° C.].

18. The process of claim 13 wherein the process is carried out at a temperature of less than about [−50° C.].

19. The process of claim 13 wherein the process is carried out at a temperature of less than about [−75° C.].
